(19) United States
(12) Patent Application Publication
(10) Pub. No.: US 2022/0002725 A1
(43) Pub. Date: Jan. 6, 2022

(54) NUCLEIC ACID-GUIDED NUCLEASE OF NICKASE FUSION EDITING OF METHYLATED NUCLEOTIDES

(71) Applicant: Inscripta, Inc., Boulder, CO (US)

(72) Inventors: Stephen Tanner, Boulder, CO (US); Michael Graige, Boulder, CO (US); Nandini Krishnamurthy, Boulder, CO (US); Craig Struble, Boulder, CO (US)

(21) Appl. No.: 17/363,884

(22) Filed: Jun. 30, 2021

Related U.S. Application Data
(60) Provisional application No. 63/046,694, filed on Jul. 1, 2020.

(51) Int. Cl. C12N 15/113; C12N 9/22; C12N 15/85

(52) U.S. Cl. CPC C12N 15/113; C12N 2310/20; C12N 15/85; C12N 9/22

(57) ABSTRACT

The present disclosure relates to compositions, methods, modules and automated, integrated instrumentation to enable nucleic acid-guided nuclease or nickase fusion editing in cells and correlating the edits to the resulting cellular nucleic acid profile. In some embodiments, methylated bases in a repair template are substituted for unmethylated bases in the cellular target genome and in some embodiments, unmethylated bases are substituted for methylated bases in the cellular target genome.

### RELATED CASES

This application claims priority to U.S. Ser. No. 63/046,694, filed 1 Jul. 2020, entitled “NUCLEIC ACID-GUIDED NUCLEASE EDITING OF METHYLATED NUCLEOTIDES”, which is incorporated herein in its entirety.

### FIELD OF THE INVENTION

The present disclosure relates to compositions, methods, modules and automated, integrated instrumentation to enable nucleic acid-guided nuclease or nickase fusion editing of methylated nucleic acid sequences and correlating the edits to the resulting cellular nucleic acid profile.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate that the articles and methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequences; hence gene function. The nucleases include nucleic acid-guided nucleases, which enable researchers to generate permanent edits in live cells, in addition to editing a nucleotide sequence, it is of interest to probe the cellular consequences of changes in DNA methylation patterns in the genome, which can change the activity of a DNA segment without changing the actual sequence of nucleotides.

There is thus a need in the art of nucleic acid-guided nuclease or nickase fusion editing for improved methods, compositions, modules and automated, integrated instruments for altering the methylation patterns in cellular genomes and determining the resulting cellular consequences. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, modules and automated multi-module cell processing instruments that allow one to perform nucleic acid-guided nuclease or nickase fusion editing of methylated nucleic acid sequences, and, further, to correlate the edits to the resulting cellular nucleic acid profile. The methods described herein include both adding methylated nucleotides into a genomic or episomic cellular sequence and replacing methylated nucleotides in a genomic or episomic cellular sequence with unmethylated nucleotides.

Thus, there is provided a method for editing a population of live cells with rationally-designed genome edits by replacing methylated nucleotide residues in genomes of the live cells with unmethylated residues or replacing unmethylated residues in genomes of the live cells with methylated residues and correlating the rationally-designed genome edits with resulting cellular nucleic acid profiles from individual cells in the population, wherein the method comprises the steps of: designing and synthesizing a library of editing cassettes wherein each editing cassette comprises a repair template and a gRNA, wherein in some editing cassettes the repair template replaces genomic methylated nucleotide residues in genomes of the live cells with unmethylated residues and wherein in some editing cassettes the repair template replaces genomic unmethylated nucleotide residues in genomes of the live cells with methylated residues; inserting the library of editing cassettes in a vector backbone resulting in a library of editing vectors; transforming the population of cells with the library of editing vectors to produce transformed cells; singulating the transformed cells into partitions; allowing editing to take place in the singulated cells to produce edited cells; lysing the edited cells; conducting bisulfite conversion to convert unmethylated cytosine residues to uracil residues in the lysed cells; adding barcoded random capture primers and barcoded cassette capture primers to each partition, wherein the barcodes used in the barcoded random capture primers and barcoded cassette capture primers in a same partition are a same barcode and wherein the barcodes used in the barcoded random capture primers and barcoded cassette capture primers in a different partition are different from barcodes used in other partitions; creating DNA copies and/or cDNAs from cellular nucleic acids in the edited cells using the barcoded random capture primers; creating DNA copies and/or cDNAs from the editing cassettes in the edited cells using the barcoded cassette capture primers; pooling the DNA copies and/or cDNAs from the partitions; sequencing the DNA copies and/or cDNAs; correlating sequences from the DNA copies and/or cDNAs from cellular nucleic acids with sequences from the DNA copies and/or cDNAs from the editing cassettes; and comparing the sequences from the DNA copies and/or cDNAs from cellular nucleic acids with a reference sequence to determine which cytosine residues in the cellular nucleic acids were converted to uracil residues for each cell.

In some aspects of this method, the sequencing step is performed by next generation sequencing, and in some aspects, the live cells are grown in an automated cell processing instrument.

In some aspects, the live cells are grown in a rotating growth module, a tangential flow filtration module, or a bioreactor module. In some aspects, the live cells are also transformed in the bioreactor module. In some aspects, the live cells are grown and transformed on microcarriers; alternatively, the live cells are grown in Accellta™ medium.

In some aspects, the cells are singulated into droplets having barcoded random capture primers and barcoded cassette capture primers and in other aspects, the cells are singulated into wells having barcoded random capture primers and barcoded cassette capture primers.

In some aspects, the cells are singulated into wells and in some aspects, the wells are in a solid wall isolation incubation and normalization (SWIIN) module.

In some aspects, the live cells are mammalian cells, and in some aspects, the mammalian cells are iPSCs or primary cells.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simple process diagram for performing nucleic acid-guided nuclease or nickase fusion editing in a population of cells to alter the methylation pattern of the genome in the cells, followed by determining the resulting cellular nucleic acid profile. FIG. 1B is a simplified depiction of the process of FIG. 1A. FIG. 1C depicts the process of bisulfite conversion of unmethylated cytosine residues to uracil residues leaving methylated cytosine residues unaffected. FIG. 1D is a depiction of the processes of reverse transcription and template switching for cellular nucleic acids in a cell after random nucleic acid primers and barcoded template switching oligonucleotides have been added to the lysate of an edited cell. FIG. 1E is a depiction of the processes of reverse transcription and template switching for editing cassette transcripts in a cell after cassette capture primers and barcoded template switching oligonucleotides have been added to the lysate of an edited cell. FIG. 1F is a depiction of the process of DNA amplification of the nucleic acids resulting from the cellular nucleic acids and editing cassette extended transcripts. FIG. 1G is a depiction of size selection of the nucleic acids resulting from the cellular nucleic acid and editing cassette extended transcripts. FIGS. 1H-A and 1H-B together are a depiction of sequencing library generation for the nucleic acids resulting from the cellular nucleic acid and editing cassette extended transcripts where sample indices and P5 and P7 sequencing primer sequences are added to the nucleic acids. FIG. 1I is a depiction of the process of reverse transcription and template switching for mRNA transcripts in a cell after poly-dT primers and barcoded template switching oligonucleotides have been added to the lysate of an individual cell.

FIGS. 2A-2C depict three different views of an exemplary automated multi-module cell processing instrument for performing nucleic acid-guided nuclease or nickase fusion editing.

FIG. 3A depicts one embodiment of a rotating growth vial for use with the cell growth module described herein and in relation to FIGS. 3B-3D. FIG. 3B illustrates a perspective view of one embodiment of a rotating growth vial in a cell growth module housing. FIG. 3C depicts a cut-away view of the cell growth module from FIG. 3B. FIG. 3D illustrates the cell growth module of FIG. 3B coupled to LED, detector, and temperature regulating components.

FIG. 4A depicts retentate (top) and permeate (bottom) members for use in a tangential flow filtration module (e.g., cell growth and/or concentration module), as well as the retentate and permeate members assembled into a tangential flow assembly (bottom).

FIG. 4B depicts two side perspective views of a reservoir assembly of a tangential flow filtration module. FIGS. 4C-4E depict an exemplary top, with fluidic and pneumatic ports and gasket suitable for the reservoir assemblies shown in FIG. 4B.

FIG. 5A depicts an exemplary combination reagent cartridge and electroporation device (e.g., transformation module) that may be used in a multi-module cell processing instrument. FIG. 5B is a top perspective view of one embodiment of an exemplary flow-through electroporation device that may be part of a reagent cartridge. FIG. 5C depicts a bottom perspective view of one embodiment of an exemplary flow-through electroporation device that may be part of a reagent cartridge. FIGS. 5D-5F depict a top perspective view, a top view of a cross section, and a side perspective view of a cross section of an FTEP device useful in a multi-module automated cell processing instrument such as that shown in FIGS. 2A-2C.

FIG. 6A depicts a simplified graphic of a workflow for singulating, editing, normalizing, and back-end analysis of cells in a solid wall device. FIGS. 6B-6D depict an embodiment of a solid wall isolation incubation and normalization (SWIIN) module.

FIG. 6E depicts the embodiment of the SWIIN module in FIGS. 6B-6D further comprising a heater and a heated cover.

FIGS. 7A-7G depict various components of an embodiment of a bioreactor useful for growing and transducing mammalian cells by the methods described herein.

FIGS. 7H-1 and 7H-2 depict an exemplary fluidic diagram for the bioreactor described in relation to FIGS. 7A-7G. FIG. 7I depicts an exemplary control system block diagram for the bioreactor described in relation to FIGS. 7A-7G.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; Chang, et al., Guide to Electroporation and Electrofusion, Academic Press, California (1992); Viral Vectors (Kaplift & Loewy, eds., Academic Press 1995), all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza & Atala, eds., Academic Press 2013); and Handbook of Stem Cells, (Atala & Lanza, eds., Academic Press 2012), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

As used herein, the terms “amplify” or “amplification” and their derivatives, refer to any operation or process whereby at least a portion of a nucleic acid molecule is replicated or copied into at least one additional nucleic acid molecule. The additional nucleic acid molecule may include a sequence that is substantially identical or substantially complementary to at least a portion of the template nucleic acid molecule. The template nucleic acid molecule can be single-stranded or double-stranded, and the additional nucleic acid molecule can be independently single-stranded or double-stranded. Amplification may include linear or exponential replication of a nucleic acid molecule. In certain embodiments, amplification can be achieved using isothermal conditions; in other embodiments, amplification may include thermocycling. In certain embodiments, the amplification is a multiplex amplification and includes the simultaneous amplification of a plurality of target sequences in a single reaction or process. In certain embodiments, “amplification” includes amplification of at least a portion of DNA and RNA based nucleic acids. The amplification reaction(s) can include any of the amplification processes known to those of ordinary skill in the art. In certain embodiments, the amplification reaction(s) includes methods such as polymerase chain reaction (PCR), ligase chain reaction (LCR), or other methods.

The term “barcoded cassette capture primer” refers to a cassette capture primer that comprises a barcode or a cassette capture primer that does not itself comprise a barcode but is combined with a barcoded template switching oligonucleotide where the combination captures and barcodes an editing cassette transcript. A “cassette capture primer” comprises a cassette capture sequence and a primer sequence.

The term “barcoded random capture primer” refers to a capture primer that comprises a barcode or a product capture primer that does not itself comprise a barcode but is combined with a barcoded template switching oligonucleotide where the combination captures and barcodes cellular nucleic acids. A barcoded random capture primer comprises a randomer sequence (e.g., random n-mer), which functions to capture most nucleic acids present in a sample (e.g., cell).

The term “capture sequence” herein refers to a nucleotide sequence that hybridizes to a nucleotide sequence of interest. The capture sequence may be, in the context of capturing cellular nucleic acids generally, random (e.g., random n-mer) hybridization sequences and the capture sequence may be, in the context of capturing an editing cassette a sequence complementary to a sequence present in an editing cassette.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “editing cassette”, “CREATE cassette”, “CREATE editing cassette”, “CREATE fusion editing cassette” or “CFE editing cassette” refers to a nucleic acid molecule comprising a coding sequence for transcription of a guide nucleic acid or gRNA covalently linked to a coding sequence for transcription of a repair template.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease or nickase fusion enzyme.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the term “nickase fusion” refers to a nucleic acid-guided nickase-(or nucleic acid-guided nuclease or CRISPR nuclease) that has been engineered to act as a nickase rather than a nuclease (e.g., the nickase portion of the fusion functions as a nickase as opposed to a nuclease that initiates double-stranded DNA breaks), where the nickase is fused to a reverse transcriptase, which is an enzyme used to generate cDNA from an RNA template. For information regarding nickase-RT fusions see, e.g., U.S. Pat. No. 10,689,669 and U.S. Ser. No. 16/740,421.

“Nucleic acid-guided editing components” refers to one, some, or all of a nucleic acid-guided nuclease or nickase fusion enzyme, a guide nucleic acid and a repair template.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein the term “repair template” refers to nucleic acid that is designed to introduce a DNA sequence modification (insertion, deletion, substitution) into a locus by homologous recombination using nucleic acid-guided nucleases or a nucleic acid that serves as a template (including a desired edit) to be incorporated into target DNA by reverse transcriptase in a nickase fusion editing system.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The term “specifically binds” as used herein includes an interaction between two molecules, e.g., an engineered peptide antigen and a binding target, with a binding affinity represented by a dissociation constant of about 10⁻⁷ M, about 10⁻⁸ M, about 10⁻⁹ M, about 10⁻¹⁰ M, about 10⁻¹¹ M, about 10⁻¹² M, about 10⁻¹³ M, about 10⁻¹⁴ M or about 10⁻¹⁵ M.

The terms “target genomic DNA sequence”, “cellular target sequence”, “target sequence”, “target cellular locus” or “genomic target locus” refer to any locus in vitro or in vivo, or in a nucleic acid (e.g., genome or episome) of a cell or population of cells, in which a change of at least one nucleotide is desired using a nucleic acid-guided nuclease or nickase fusion editing system. The target sequence can be a genomic locus or extrachromosomal locus. The term “edited target sequence” or “edited locus” refers to a target genomic sequence or target sequence after editing has been performed, where the edited target sequence comprises the desired edit.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In some embodiments of the present methods, two vectors—an engine vector, comprising the coding sequences for a nuclease or nickase fusion, and an editing vector, comprising the gRNA sequence and the repair template—are used. In alternative embodiments, all editing components, including the nuclease or nickase fusion, gRNA sequence, and repair template sequence are all on the same vector (e.g., a combined editing/engine vector).

### Nuclease-Directed Genome Editing Generally

The compositions, methods, modules and instruments described herein are employed to allow one to perform nucleic acid nuclease-directed genome editing to introduce desired edits to a population of live cells and then allow one to quickly identify edited cells in vivo. Specifically, the compositions, methods, modules and integrated instruments presented herein enable nucleic acid-guided nuclease or nickase fusion editing of methylated nucleic acid sequences to produce a different methylation profile in a cellular genome followed by correlating the methylation profile to the resulting cellular nucleic acid profile (e.g., mRNA profile or transcriptome). A nucleic acid-guided nuclease or nickase fusion complexed with an appropriate synthetic guide nucleic acid in a cell can cut the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion may be programmed to target any DNA sequence for cleavage as long as an appropriate protospacer adjacent motif (PAM) is nearby. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion enzyme.

Generally, a nucleic acid-guided nuclease or nickase fusion complexed with an appropriate synthetic guide nucleic acid in a cell can cut the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion may be programmed to target any DNA sequence for cleavage as long as an appropriate protospacer adjacent motif (PAM) is nearby. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion.

In general, a guide nucleic acid (e.g., gRNA) complexes with a compatible nucleic acid-guided nuclease or nickase fusion and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. Preferably and typically, the guide nucleic acid comprises RNA and the gRNA is encoded by a DNA sequence on an editing cassette along with the coding sequence for a repair template. Covalently linking the gRNA and repair template allows one to scale up the number of edits that can be made in a population of cells tremendously. Methods and compositions for designing and synthesizing editing cassettes (e.g., CREATE cassettes) are described in U.S. Pat. Nos. 10,240,167; 10,266,849; 9,982,278; 10,351,877; 10,364,442; 10,435,715; 10,669,559; 10,711,284; 10,731,180, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

The guide nucleic acid may be and preferably is part of an editing cassette that encodes the repair template that targets a cellular target sequence. Alternatively, the guide nucleic acid may not be part of the editing cassette and instead may be encoded on the editing vector backbone. For example, a sequence coding for a guide nucleic acid can be assembled or inserted into a vector backbone first, followed by insertion of the repair template in, e.g., an editing cassette. In other cases, the repair template in, e.g., an editing cassette can be inserted or assembled into a vector backbone first, followed by insertion of the sequence coding for the guide nucleic acid. Preferably, the sequence encoding the guide nucleic acid and the repair template are located together in a rationally-designed editing cassette and are simultaneously inserted or assembled via gap repair into a linear plasmid or vector backbone to create an editing vector.

The target sequence is associated with a proto-spacer mutation (PAM), which is a short nucleotide sequence recognized by the gRNA/nuclease or nickase fusion complex. The precise preferred PAM sequence and length requirements for different nucleic acid-guided nucleases and or nickase fusions vary; however, PAMs typically are 2-7 base-pair sequences adjacent or in proximity to the target sequence and, depending on the nuclease or nickase fusion, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most embodiments, genome editing of a cellular target sequence both introduces a desired DNA change to a cellular target sequence, e.g., the genomic DNA of a cell, and removes, mutates, or renders inactive a proto-spacer mutation (PAM) region in the cellular target sequence (e.g., thereby rendering the target site immune to further nuclease or nickase fusion binding). Rendering the PAM at the cellular target sequence inactive precludes additional editing of the cell genome at that cellular target sequence, e.g., upon subsequent exposure to a nucleic acid-guided nuclease or nickase fusion complexed with a synthetic guide nucleic acid in later rounds of editing. Thus, cells having the desired cellular target sequence edit and an altered PAM can be selected for by using a nucleic acid-guided nuclease or nickase fusion complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or nickase fusion component of the nucleic acid-guided nuclease or nickase fusion editing system, a polynucleotide sequence encoding the nucleic acid-guided nuclease or nickase fusion can be codon optimized for expression in particular cell types, such as bacterial, yeast, and mammalian cells. The choice of the nucleic acid-guided nuclease or nickase fusion to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. Nickase fusion enzymes of use in the methods described herein include but are not limited to nickase fusion enzymes developed from Cas 9, Cas 12/CpfI, MAD2, or MAD7 or other MADzymes.

Another component of the nucleic acid-guided nuclease of use in the methods described herein include but are not limited to Cas 9, Cas 12/CpfI, MAD2, or MAD7 or other MADzymes system is the repair template comprising homology to the cellular target sequence. For the present methods and compositions, the repair template is on the same vector and in the same editing cassette as the guide nucleic acid and is under the control of the same promoter as the editing gRNA (that is, a single promoter driving the transcription of both the editing gRNA and the repair template). The repair template is designed to serve as a template for homologous recombination with a cellular target sequence nicked or cleaved by the nucleic acid-guided nuclease of use in the methods described herein include but are not limited to Cas 9, Cas 12/CpfI, MAD2, or MAD7 or other MADzymes as a part of the gRNA/nuclease or nickase fusion complex. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Ser. No. 16/275,465, filed 14 Feb. 2019. In certain preferred aspects, the repair template can be provided as an oligonucleotide of between 20-300 nucleotides, more preferably between 50-250 nucleotides. The repair template comprises a region that is complementary to a portion of the cellular target sequence (e.g., a homology arm). When optimally aligned, the repair template overlaps with (is complementary to) the cellular target sequence by, e.g., about 20, 25, 30, 35, 40, 50, 60, 70, 80, 90 or more nucleotides. In many embodiments, the repair template comprises two homology arms (regions complementary to the cellular target sequence) flanking the mutation or difference between the repair template and the cellular target sequence. The repair template comprises at least one mutation or alteration compared to the cellular target sequence, such as an insertion, deletion, modification, or any combination thereof compared to the cellular target sequence.

As described in relation to the gRNA, the repair template is provided as part of a rationally-designed editing cassette, which is inserted into an editing plasmid backbone (in yeast, preferably a linear plasmid backbone) where the editing plasmid backbone may comprise a promoter to drive transcription of the editing gRNA and the repair template when the editing cassette is inserted into the editing plasmid backbone. Moreover, there may be more than one, e.g., two, three, four, or more editing gRNA/repair template rationally-designed editing cassettes inserted into an editing vector; alternatively, a single rationally-designed editing cassette may comprise two to several editing gRNA/repair template pairs, where each editing gRNA is under the control of separate different promoters, separate like promoters, or where all gRNAs/repair template pairs are under the control of a single promoter. In some embodiments the promoter driving transcription of the editing gRNA and the repair template (or driving more than one editing gRNA/repair template pair) is optionally an inducible promoter.

In addition to the repair template, an editing cassette may comprise one or more primer binding sites. The primer binding sites are used to amplify the editing cassette by using oligonucleotide primers as described infra and may be biotinylated or otherwise labeled. In addition, the editing cassette may comprise a barcode. A barcode is a unique DNA sequence that corresponds to the repair template sequence such that the barcode can identify the edit made to the corresponding cellular target sequence. The barcode typically comprises four or more nucleotides. In some embodiments, the editing cassettes comprise a collection or library editing gRNAs and of repair templates representing, e.g., gene-wide or genome-wide libraries of editing gRNAs and repair templates. The library of editing cassettes is cloned into vector backbones where, e.g., each different repair template is associated with a different barcode. Also, in preferred embodiments, an editing vector or plasmid encoding components of the nucleic acid-guided nuclease or nickase fusion system further encodes a nucleic acid-guided nuclease or nickase fusion comprising one or more nuclear localization sequences (NLSs), such as about or more than about 1, 2, 3, 4, 5, 6, 7, 8, 9, 10, or more NLSs, particularly as an element of the nuclease or nickase fusion sequence. In some embodiments, the engineered nuclease or nickase fusion comprises NLSs at or near the amino-terminus, NLSs at or near the carboxy-terminus, or a combination.

Editing Cells to Alter Methylation Patterns in Cells and Correlating the Altered Methylation Patterns with the Resulting Cellular Nucleic Acid Profiles

The present disclosure is drawn to methods, compositions, modules and automated, integrated instruments that allow editing of live cells to create a change in genomic or episomic nucleic acids and to then correlate the genome edit or change with the cellular nucleic acid profile resulting from that change. The methods described herein include adding methylated nucleotides into a genomic or episomic cellular target sequence and/or replacing methylated nucleotides in a genomic or episomic cellular target sequence with unmethylated nucleotides. The present methods allow for multiplex editing in a large population of cells using a library of different rationally-designed editing vectors where each editing vector comprises a different editing cassette. Once the edits have taken place, there are also provided methods for correlating the resulting edit(s) (via DNA copies or cDNAs of the editing cassettes) to the resulting cellular nucleic acid profile (e.g., transcriptome) of selected or all nucleic acids in the cell.

In nature, DNA methylation is a biological process by which methyl groups are added to a DNA molecule. Methylation can change the activity of a DNA segment without changing the nucleotide sequence. When located in a gene promoter, DNA methylation typically acts to repress gene transcription. Two of DNA's four bases, cytosine and adenine, can be methylated. Cytosine methylation is widespread in both eukaryotes and prokaryotes, even though the rate of cytosine DNA methylation can differ greatly between species. DNA methylation in vertebrates is mainly restricted to CpG sites, but significant non-CpG methylation has been found in pluripotent stem cells. There are approximately 29 million CpGs in the human genome and 60-80% of them are methylated. Approximately 7% of CpGs are located in CpG islands, which are regions of high CG density. Approximately 70% of annotated gene promoters are associated with a CGI and CGIs are largely resistant to DNA methylation. The enzymes responsible for DNA methylation are DNA methyltransferases (DNMTs) including DNMT1, DNMT3A, DNMT3B and DNMT3C.

In recent decades, researchers have learned a great deal about DNA methylation, including how it occurs and where it occurs, and they have also discovered that methylation is an important component in numerous cellular processes, including embryonic development, genomic imprinting, X-chromosome inactivation, repression of transposable elements, aging, carcinogenesis and preservation of chromosome stability. Given the many processes in which methylation plays a part, it is perhaps not surprising that errors in methylation have been linked to a variety of devastating consequences, including several human diseases.

Editing typically involves making a cut (double-stranded break or single-stranded nick) in a host cell DNA, followed by a paste, which is a repair mediated by repair template (e.g., homology arms) in homology-directed repair, random repair mediated by non-homologous end joining, or primed replication. The DNA bases of a repair template in an editing cassette produced using chemical oligonucleotide synthesis are typically not methylated, although specialty bases may be used to impart particular methylation patterns. Therefore, editing cassettes may be engineered to have unmethylated or differentially-methylated states compared to the methylation pattern of the native cellular DNA target sequence which is replaced during the editing process. In the instance where a methylated residue in the cellular genome is replaced with an unmethylated residue, immediately after editing the cellular target sequence in the genome is “reset” to an unmodified state; likewise, in the instance where an unmethylated residue in the cellular genome is replaced with a methylated residue, immediately after editing the cellular target sequence in the genome is “reset” to a modified state. The ability to reset the genomic methylation state through CRISPR editing is valuable for probing gene regulation and may be enhanced further by using editing cassettes in conjunction with a CRISPR-directed methylase or other enzyme directing epigenetic modifications, to enable adding—as well as removing—epigenetic modifications across a wide selection of cellular target sequences.

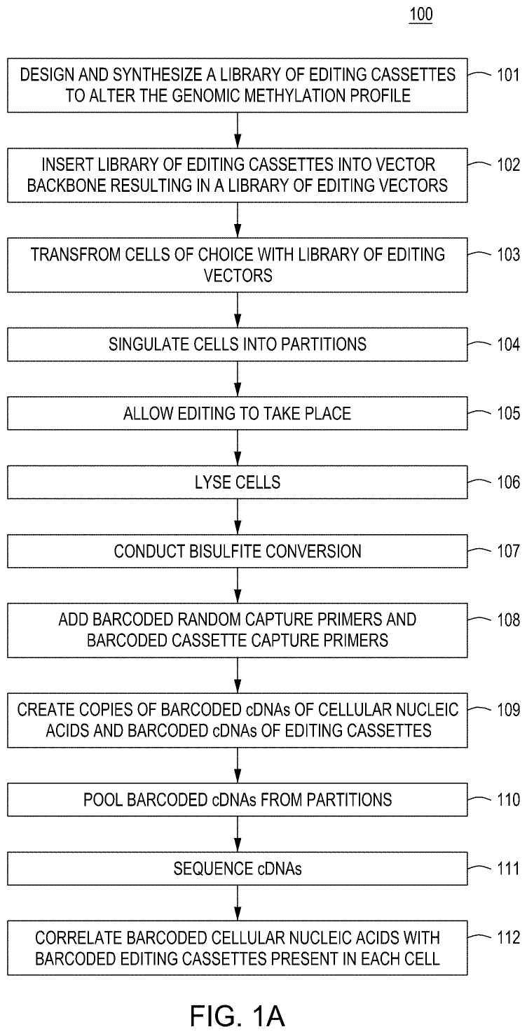

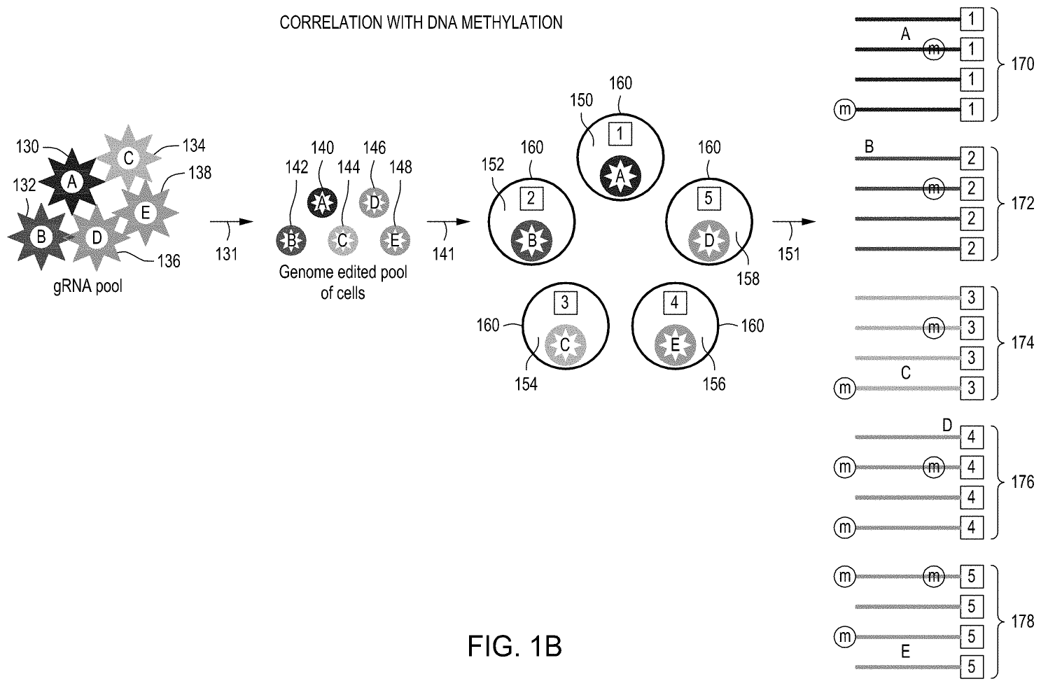

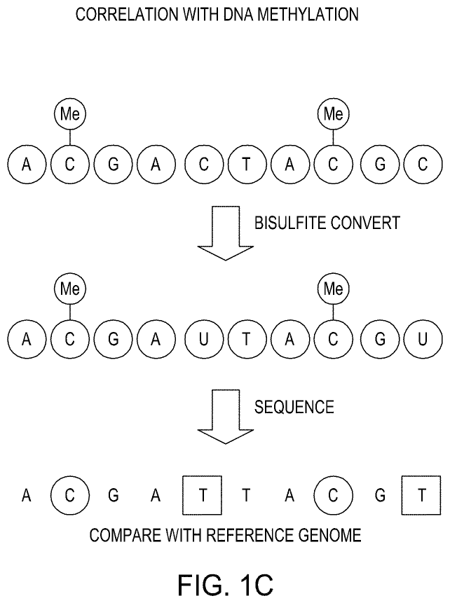

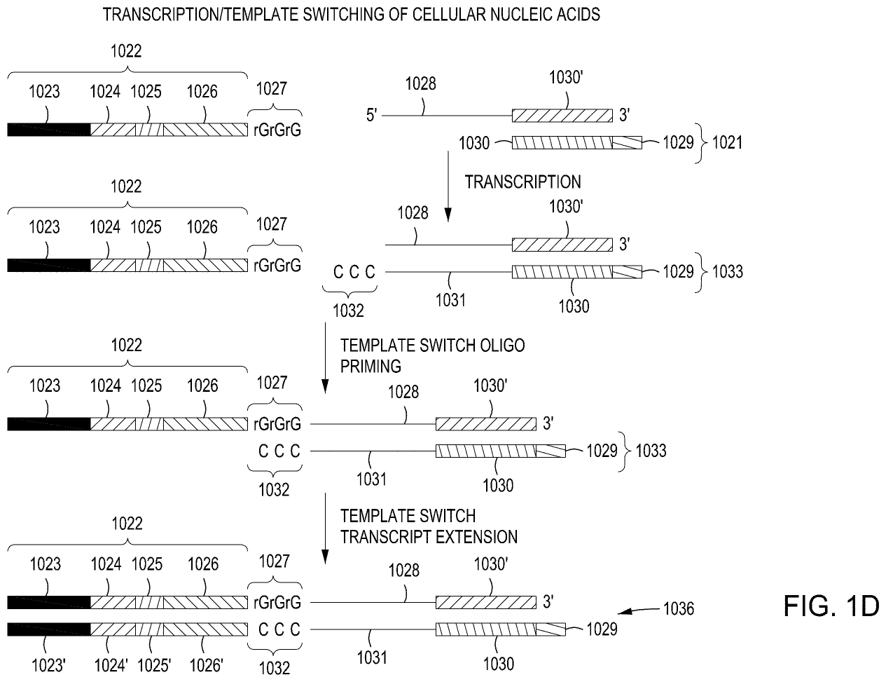

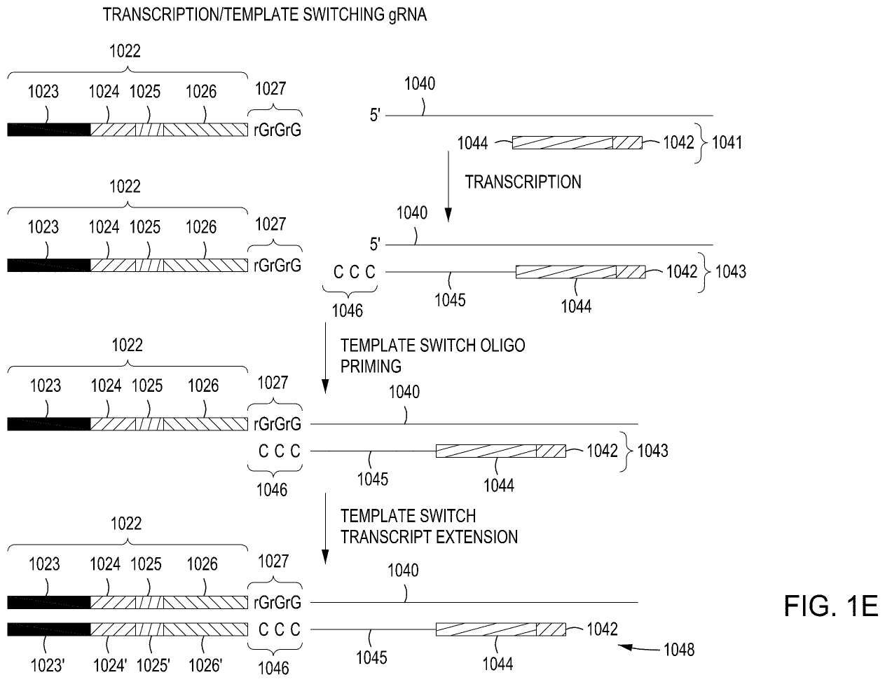

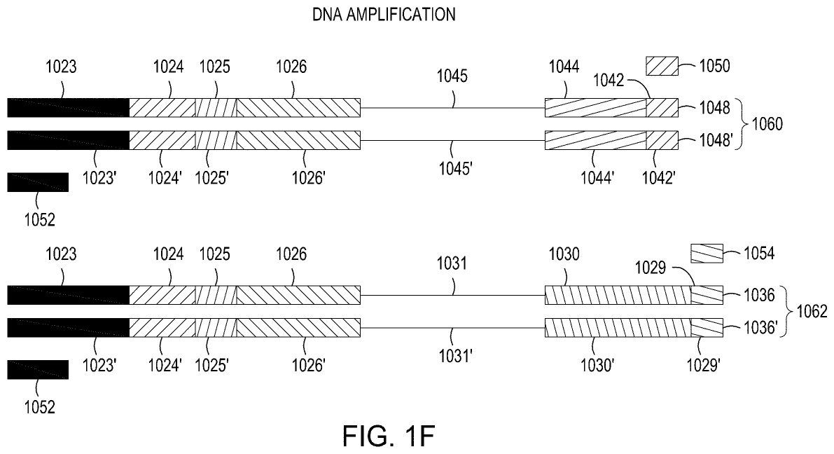

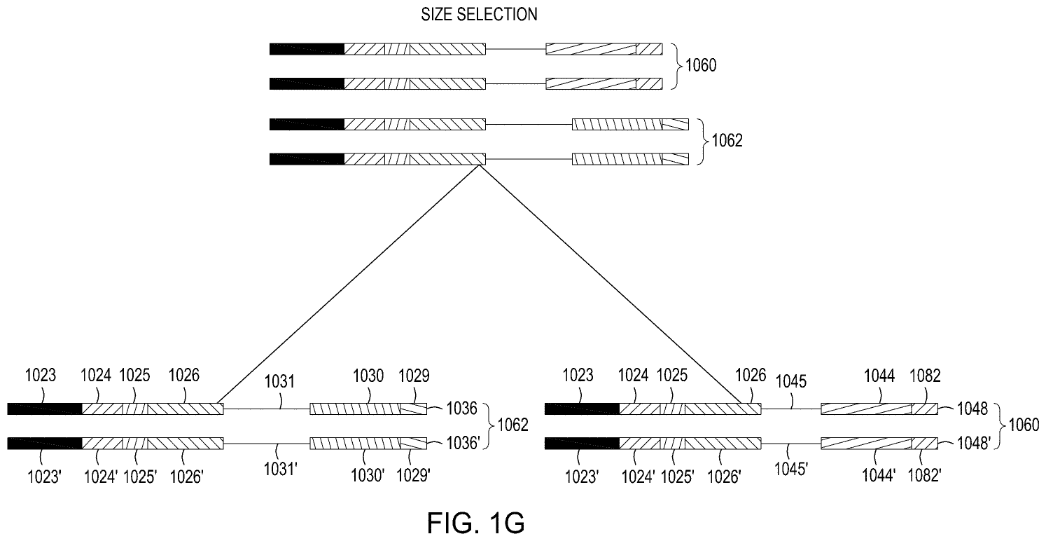

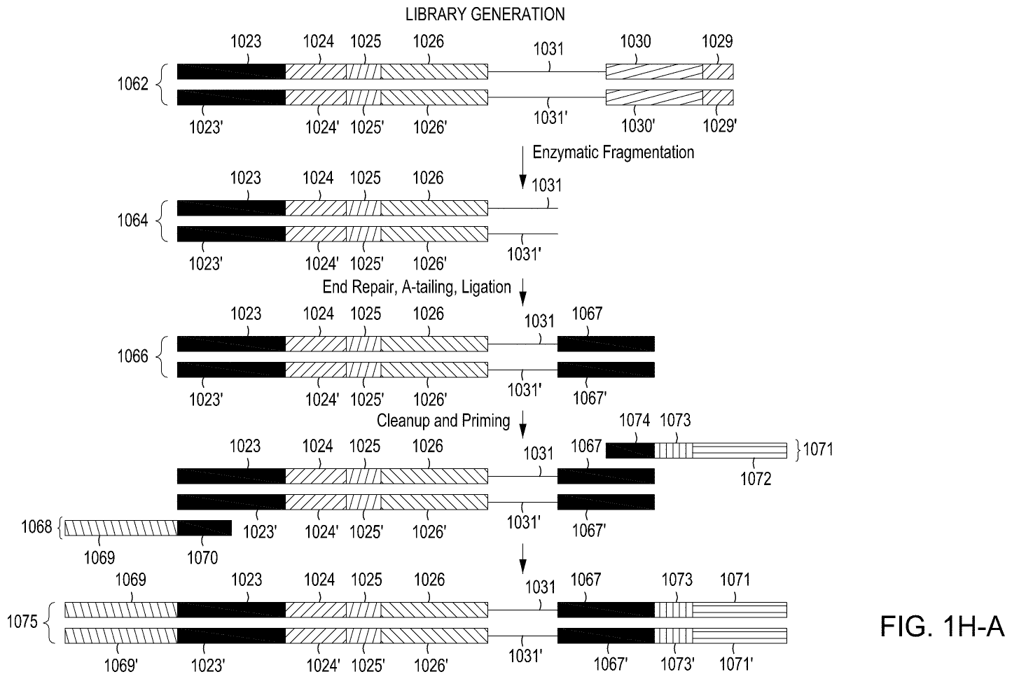

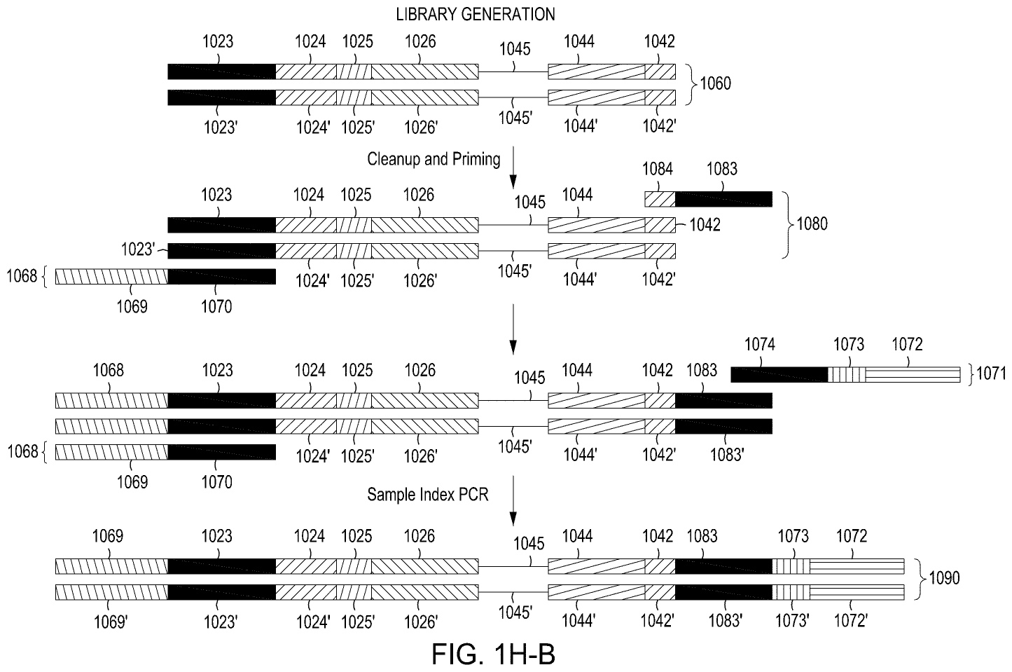

FIG. 1A is a simple process diagram for multiplexed nucleic acid-guided nuclease or nickase fusion editing in a population of cells and determining the genomic DNA methylation profile resulting from one or more edits in individual cells in the population using barcoded cassette capture primers to capture editing cassettes and barcoded product capture primers (here, barcoded random capture primers) to capture nucleic acids from each cell. In a first step 101 of method 100, a library of editing cassettes comprising paired gRNAs and repair templates is designed and synthesized. In addition to paired gRNAs and repair templates, the editing cassettes preferably comprise additional sequences such as one or more priming sequences that can be used to amplify the editing cassette; an editing cassette barcode, which is used to uniquely identify the intended edit to be made by the gRNA and repair template pair; and/or a capture sequence, where the capture sequence facilitates capture of the editing cassette by the cassette capture primers when analyzing the edit/DNA methylation profile relationship. To add methylated bases in a genome, a CRISPR-directed methylase may be used.

Once designed and synthesized 101, the library of editing cassettes is amplified, purified and inserted 102 into a vector backbone—which in some embodiments may already comprise a coding sequence for the nuclease or nickase fusion—to produce a library of editing vectors. Alternatively, the coding sequence for the nuclease or nickase fusion may be located on another vector or may be integrated into the cellular genome. In yet another alternative, the nuclease or nickase fusion may be delivered to the cell as a protein. The vectors chosen for the methods herein will vary depending on the type of cells being edited and analyzed, where the vectors include, e.g., plasmids, BACs, YACs, viral vectors and synthetic chromosomes.

The cells of interest useful in the methods herein are any cells, including bacterial, yeast and animal (including mammalian) cells. Bacterial genomes are known to contain methylated bases as are lower eukaryotic, e.g., yeast genomes; however, of primary interest are mammalian cells. Before being transformed by the editing vectors, the cells are often grown in culture for several passages. Cell culture is the process by which cells are grown under controlled conditions, almost always outside the cell's natural environment. For mammalian cells, culture conditions typically vary somewhat for each cell type but generally include a medium and additives that supply essential nutrients such as amino acids, carbohydrates, vitamins, minerals, growth factors, hormones, and gases such as, e.g., O₂ and CO₂. In addition to providing nutrients, the medium typically regulates the physio-chemical environment via a pH buffer and most cells are grown at 37° C. Many mammalian cells require or prefer a surface or artificial substrate on which to grow (e.g., adherent cells), whereas other cells such as hematopoietic cells and some adherent cells can be grown in or adapted to grow in suspension. Adherent cells often are grown in 2D monolayer cultures in petri dishes or flasks, but some adherent cells can grow in suspension cultures to higher density than would be possible in 2D cultures. “Passages” generally refers to transferring a small number of cells to a fresh substrate with fresh medium, or, in the case of suspension cultures, transferring a small volume of the culture to a larger volume of medium.

The cells of choice are provided and are transformed with the library of editing vectors 103. Transformation is intended to generically include a variety of art-recognized techniques for introducing an exogenous nucleic acid sequence (e.g., an engine and/or editing vector) into a target cell, and the term “transformation” as used herein includes all transformation and transfection techniques. Such methods include, but are not limited to, electroporation, lipofection, optoporation, injection, croprecipitation, r Microinjection, liposomes. particle bombardment, sonoporation, laser-induced poration, bead transfection. calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th, ed., Cold Spring Harbor Laboratory Press, Cold Spring Harbor, N.Y., 2014).

Once transformed 103, the cells are allowed to recover and selection optionally is performed to select for cells transformed with the editing vector, which most often comprises a selectable marker. At a next step 104, the cells are singulated or partitioned into partitions (e.g., wells, but also droplets or beads) where there is approximately 1 cell per two wells (e.g., a Poisson distribution of cells). After singulation, conditions are provided such that editing takes place and the cells are incubated to allow for transcription and translation of nucleic acids and proteins, respectively 105.

Once editing has taken place, the cells are lysed in situ 106. After the cells are lysed 107 to release the nucleic acids present in each cell, the cellular nucleic acids are treated to “bisulfite conversion” 107. Bisulfite conversion is a process in which genomic DNA is denatured and treated with sodium bisulfite, leading to deamination of unmethylated cytosines into uracils, while methylated cytosines (both 5-methylcytosine and 5-hydroxymethylcytosine) remain unchanged. The DNA is then amplified by PCR where the uracils are converted to thymines. Bisulfite converted DNA can be analyzed for gene- or allele-specific methylation patterns by comparing the sequence of the converted DNA to untreated DNA creating a methylation profile of the sample. The results provide single nucleotide resolution information about the methylation status. FIG. 1C, described infra, depicts this process. As an alternative to bisulfite conversion, TET-assisted pyridine borane sequencing (TAPS) may be employed. TAPs detects both 5-methylcytosine and 5-hydroxymethylcytosine with high sensitivity and specificity without affecting unmodified cytosines. (See Liu, et al., Nat. Biotech., 37:424-29 (2019).)

Once the cellular nucleic acids in the lysate have been converted by bisulfite conversion 107, barcoded random capture primers (or the random capture primers and barcoded template switching oligonucleotides as described in detail in relation to FIGS. 1D-1E) and barcoded cassette capture primers (or the cassette capture primers and barcoded template switching oligonucleotides as described in detail in relation to FIGS. 1D-1E) are added 108. The template switching oligonucleotides in each partition comprise a different, unique cellular barcode and thus the nucleic acids from the cells in each partition are labeled with this unique cellular barcode. After the barcoded random capture primers and barcoded cassette capture primers are added, DNA copies or cDNAs are created from the cellular nucleic acids and from the editing cassettes present in the cell 109 using a combination of one or more of the processes of priming, reverse transcription or transcription, extension and amplification. An exemplary process for creating DNA or cDNA copies of cellular nucleic acids and editing cassettes is shown in FIGS. 1D-1E.

After the DNA copies are synthesized, they are pooled 110 and sequenced 111. Because each partition comprised barcoded cassette capture primers and barcoded random capture primers with a unique cellular barcode, each nucleic acid from each cellular nucleic acid and each nucleic acid from each editing cassette transcript from each cell (or cell colony) is tagged with this unique cellular barcode; thus, the nucleic acids representing the cellular nucleic acids and the nucleic acids representing the editing cassettes from each partition can be correlated 112. Correlation allows one to match adding a methylated base or subtracting a methylated base to a change in the resulting nucleic acid (e.g., mRNA) profile in the cell. Once correlated, the cellular nucleic acids are then compared to, e.g., a reference sequence so as to determine which cytosine residues were not methylated. Unmethylated cytosines will appear as thymine residues (or adenine complement) upon bisulfite conversion and DNA copy or cDNA creation.

It should be apparent to one of ordinary skill in the art given the present disclosure that the multiplexed library of editing cassettes (and the editing vectors comprising the editing cassettes) that some editing cassettes in a library may replace methylated bases in the target genome sequence, some editing cassettes in the same library may replace unmethylated bases in the target genome sequence, and some editing cassettes in the same library may impart other types of edits in the target genome sequence, including swaps, insertions, and deletions.

FIG. 1B is a simplified depiction of the process of FIG. 1A. At left in FIG. 1B is a pool of editing vectors (130, 132, 134, 136, and 138), where each editing vector comprises a different editing cassette (e.g., a gRNA and repair template pair) and the editing cassette optionally comprises a barcode that uniquely identifies the intended edit to be made by the gRNA and repair template pair. If the editing cassette does not comprise a unique barcode, the editing cassette itself serves as the “barcode.” At step 131 a population of cells is transformed with the pool of editing vectors and conditions are provided to promote nucleic acid guided-nuclease or nickase fusion editing in the cells, producing a genome-edited pool of cells (e.g., cell 140 edited by editing vector 130; cell 142 edited by editing vector 132; cell 144 edited by editing vector 134; cell 146 edited by editing vector 136; and cell 148 edited by editing vector 138), in this case genome edits where at least in some cells methylated bases are replaced with unmethylated bases in the cellular genome or where unmethylated bases are replaced with methylated bases in the genome.

At step 141 the cells are singulated into partitions 160, and allowed to edit. Following editing and cell growth, the cells are lysed and treated to bisulfite conversion, which converts unmethylated cytosine residues in the cellular nucleic acids to uracil residues and leaves methylated cytosine residues untouched. Once bisulfite conversion has taken place, barcoded random capture primers and barcoded cassette capture primers (or random capture primers, cassette capture primers and barcoded template switching oligonucleotides) are added to each partition. Partition 150 comprises cell 140 with barcode 1; partition 152 comprises cell 142 with barcode 2; partition 154 comprises cell 144 with barcode 3; partition 156 comprises cell 146 with barcode 4; and partition 158 comprises cell 148 with barcode 5.

At step 151, DNA copies or cDNAs are prepared from the cellular nucleic acids and the editing cassettes, the barcoded DNA copies are pooled, and the DNA copies or cDNAs from the cellular nucleic acids are correlated with the editing cassettes present in each cell. The nucleic acids of group 170 are associated with editing cassette A, cellular barcode 1 and two nucleic acids representing cellular nucleic acid sequences with methylated cytosines; the nucleic acids of group 172 are associated with editing cassette B, cellular barcode 2 and one nucleic acid representing a cellular nucleic acid sequence with methylated cytosines; the nucleic acids of group 174 are associated with editing cassette C, cellular barcode 3 and two nucleic acids representing cellular nucleic acid sequences with methylated cytosines; the nucleic acids of group 176 are associated with editing cassette D, cellular barcode 4 and one nucleic acid representing a cellular nucleic acid sequence with methylated cytosines; and the nucleic acids of group 178 are associated with editing cassette E, cellular barcode 5 and one nucleic acid representing a cellular nucleic acid sequence with methylated cytosines. In yet another embodiment for correlating nucleic acid-guided editing with the resulting methylation profiles, nucleic acid-tagged antibodies against methylcytosine residues may be employed.

FIG. 1C is a simplified representation of bisulfite conversion of unmethylated cytosine residues to uracil residues. An exemplary sequence 5′-AC(me)GACTAC(me)GC-3′ is converted by bisulfite conversion into 5′-AC(me)GAUTAC(me)GU-3′ (here, the unmethylated cytosine residues that were converted into uracil residues are bolded). When sequenced, the sequence will now be read ACGATTACGT-3′. In FIG. 1C, the circled C's were methylated, and thus were not converted to uracil residues and will “remain” C's whereas the boxed T's correlate to unmethylated cytosine residues that were converted to uracil residues and when reversed transcribed and sequenced are now T's. The errant T's can be identified as C's by comparison to a reference genome.

FIG. 1D is a depiction of the processes of reverse transcription and template switching for cellular nucleic acids to create DNA copies or cDNAs (“DNA copies”) in a cell after random capture primers and template switching oligonucleotides have been combined with the lysate of an individual cell (e.g., step 110 of FIG. 1A). At top left of FIG. 1D is a template switching oligonucleotide (TSO) 1022. TSO comprises from left (5′) to right (3′) a read 1 sequencing primer binding sequence 1023, a cellular barcode 1024, a unique molecular identifier 1025 and a TSO sequence 1026 comprising a poly-dG tract 1027. Cellular barcode 1024 is unique to each partition whether the partition is a droplet, gel bead or a well, and the unique molecular identifiers 1025 comprise a tract of nucleotides coupled with a particular cellular barcode where each unique molecular identifier coupled with a particular cellular barcode is different. Cellular barcode 1024 facilitates association of DNA copies created from the cellular nucleic acids and editing cassette transcripts originating from a single cell, and the unique molecular identifiers allow tracking of DNA copies originating from a single DNA copy after amplification.

At top right of FIG. 1D is a cellular nucleic acid 1028. Below cellular nucleic acids 1028 is a random capture primer 1021 comprising a priming sequence 1029 and a random hybridization sequence 1030 that can hybridize to a complementary sequence 1030′ in cellular nucleic acid 1028. Random priming is an efficient method for copying cellular nucleic acids to produce DNA libraries. Random priming—i.e., using random sequences to hybridize to complementary sequence in the cellular genome—has been used to amplify sequences from an entire genome in a single cell. In a next step, complementary sequence in the cellular nucleic acid 1028 and random capture primer 1021 are hybridized and a copy is made 1031 cellular nucleic acid 1028 resulting in a reverse transcript construct 1033. During reverse transcription of the cellular nucleic acid several to many untemplated Cs 1032 are added to the 3′ end of the reverse transcript construct 1033. Untemplated Cs 1032 are capable of hybridizing with the poly-dG tract 1027 of TSO 1022 (allowing for, e.g., TSO priming). After TSO priming of the cellular nucleic acid 1028 and reverse transcript construct 1033, the reverse transcript construct 1033 is extended from the untemplated Cs 1032 to include TSO sequence complement 1026′, unique molecular identifier complement 1025′, cellular barcode complement 1024′, and read 1 sequencing primer binding sequence complement 1023′ resulting in an extended cDNA transcript 1036.

FIG. 1E is a depiction and exemplary embodiment of the processes of reverse transcription and template switching for editing cassette transcripts in a cell after cassette capture primers and barcoded template switching oligonucleotides have been combined with the lysate of an individual cell (e.g., step 110 of FIG. 1A). At top left of FIG. 1E is a template switching oligonucleotide (TSO) 1022. Like the TSO in FIG. 1D, the TSO comprises from left (5′) to right (3′) read 1 sequencing primer binding sequence 1023, a cellular barcode 1024, a unique molecular identifier 1025 and a TSO sequence 1026 comprising a poly-dG tract 1027. Also as with the TSO depicted in FIG. 1D, cellular barcode 1024 is unique to each partition whether the partition is a droplet or a well, and the unique molecular identifiers 1025 comprise a tract of nucleotides coupled with a particular cellular barcode where each unique molecular identifier coupled with a particular cellular barcode is different. Cellular barcode 1024 facilitates association of the DNA copies created from the cellular nucleic acids and editing cassette transcripts originating from a single cell, and the unique molecular identifiers allow tracking of DNA copies originating from a single DNA copy after amplification.

At top right of FIG. 1E is editing cassette transcript 1040 and positioned below editing cassette transcript 1040 in this FIG. 1E is cassette capture primer 1041 comprising a priming sequence 1042 and a cassette capture sequence 1044 that is complementary to a sequence associated with the editing cassette (e.g., complementary to part of the editing cassette itself or complementary to an cassette capture sequence). In a next step, the editing cassette transcript 1040 and cassette capture primer 1041 are hybridized and reverse transcription is performed primed from cassette capture primer 1041 resulting in a copy 1045 of editing cassette transcript 1040. During reverse transcription of editing cassette transcript 1040, several to many untemplated Cs 1046 are added to the 3′ end of the reverse transcript construct 1043. Untemplated Cs 1046 are capable of hybridizing with the poly-dG tract 127 of TSO 1022 (allowing for, e.g., TSO priming). After TSO priming of editing cassette transcript 1040 and reverse transcript construct 1043, reverse transcript construct 1043 is extended from the untemplated Cs 1046 to include TSO sequence complement 1026′, unique molecular identifier complement 1025′, cellular barcode complement 1024′, and read 1 sequencing primer binding sequence complement 1023′ resulting in an extended cDNA transcript 1048.

FIG. 1F is a depiction of the process of amplification of the duplex—extended DNA copies created from the cellular nucleic acids and editing cassettes. At top is a duplex 1060 of the extended DNA transcript 1048 and its complement 1048′ resulting from copying the editing cassettes present in the cells. The duplex extended nucleic acid 1048/1048′ comprises from left (5′) to right (3′) read 1 sequencing primer binding sequence 1023 (and its complement 1023′), cellular barcode 1024 (and its complement 1024′), a unique molecular identifier 1025 (and its complement 1025′), a TSO sequence 1026 (and its complement 1026′), a poly-dG tract 1027 (and its complement poly-dC tract 1046) (neither shown in this FIG. 1F), the copy of the editing cassette transcript 1045 (and its complement 1045′), editing cassette complement sequence 1044 (and its complement cassette capture sequence 1044′), and priming sequence 1042 (and its complement 1042′). Amplification primer 1050 binds to priming sequence 1042 and sequencing read amplification primer 1052 binds to the complement of read 1 sequencing primer binding sequence 1023′ to amplify the duplex extended DNA transcript 1048/1048′.

At bottom of FIG. 1F is a duplex 1062 of the extended DNA transcript 1036 and its complement 1036′ resulting from copying the nucleic acids in the cell. The duplex extended DNA transcript 636 and its complement 1036′ comprises from left (5′) to right (3′) read 1 sequencing primer binding sequence 1023 (and its complement 1023′), cellular barcode 1024 (and its complement 1024′), a unique molecular identifier 1025 (and its complement 1025′), a TSO sequence 1026 (and its complement 1026′), a poly-dG tract (and its complement poly-dC tract 1046) (neither shown in this FIG. 1F), the copy of the nucleic acid 1031 (and its complement 1031′), random hybridization sequence 1030 (and its complement 1030′, a poly-A from the mRNA transcript), and priming sequence 1029 (and its complement 1029′). Amplification primer 1054 binds to priming sequence 1036 and sequencing read amplification primer 1052 binds to the complement of read 1 sequencing primer binding sequence 1023′ to amplify extended DNA transcript 1036 and its complement 1036′.

FIG. 1G is a depiction of size selection of the cellular nucleic acids and editing cassette extended transcripts. The extended DNA transcripts 1062 created from copying the cellular nucleic acids in the cell and extended DNA transcripts 1060 created from copying the editing cassettes in the cell differ in size.

FIGS. 1H-A and 1H-B are depictions of sequencing library generation for the random cellular nucleic acid- and editing cassette-generated DNA copies where sample indices and P5 and P7 sequencing primer sequences are added to the DNA copies. In FIG. 1H-A, the size-selected DNA duplex created from the cellular nucleic acid extended transcript 1062 is seen. Size-selected DNA duplex 1062 comprises from left (5′) to right (3′) read 1 sequencing primer binding sequence 1023 (and its complement 1023′), cellular barcode 1024 (and its complement 1024′), unique molecular identifier 1025 (and its complement 1025′), TSO sequence 1026 (and its complement 1026′), a poly-dG tract (and its complement poly-dC tract 1046) (neither shown in this FIG. 1H), cellular nucleic acid transcript 1031 (and its complement 1031′), random hybridization sequence 1030 (and its complement 1030′ cellular nucleic acid transcript), and priming sequence 1029 (and its complement 1029′). Enzymatic fragmentation is then performed creating truncated DNA duplex 1064 where a portion of random cellular nucleic acid transcript 1031 (and its complement 1031′) is cleaved, thereby cleaving off a portion of the random capture primer 1030 and the cellular nucleic acid sequence complement 1030′ as well as the priming sequence 1029 (and its complement 1029′). Following enzymatic fragmentation, a combination of end repair, A-tailing and ligation of a read 2 sequencing primer binding sequence to the 3′ end of truncated DNA duplex 1064 is performed to create DNA duplex 1066 with read 1 sequencing primer binding sequence 1023 at its 5′ end and read 2 sequencing primer binding sequence 1067 at its 3′ end (with the complements thereof 1023′ and 1067′, respectively).

After ligation of the read 2 sequencing primer binding sequence 1067, 1067′, DNA duplex 1066 is primed with a P5 primer 1068 comprising a P5 sequence 1069 and a read 1 primer 1070, and with a P7/sample index primer 1071 comprising a P7 sequence 1072, a sample index 1073 and a read 2 primer 1074. Amplification with P5 primer 1068 and P7/sample index primer 1071 results in final DNA library constructs 1075 created from the cellular mRNAs ready for sequencing on ILLUMINA's HiSeq®, MiSeq®, NextSeq, NovaSeq platforms or other ILLUMINA sequencing systems. Final DNA library constructs 1075 comprise from 5′ to 3′ P5 sequence 1069 (and its complement 1069′), read 1 sequencing primer binding sequence 1023 (and its complement 1023′), cellular barcode 1024 (and its complement 1024′), unique molecular identifier 1025 (and its complement 1025′), TSO sequence 1026 (and its complement 1026′), mRNA sequence 1031 (and its complement 1031′), read 2 sequencing primer binding sequence 1067 (and its complement 1067′), sample index 1073 (and its complement 1073′), and P7 sequence 1072 (and its complement 1072′).

In FIG. 1H-B, the size-selected DNA duplex 1060 corresponding to the editing cassettes present in the cell is seen. Size-selected DNA duplex 1060 comprises from left (5′) to right (3′) read 1 sequencing primer binding sequence 1023 (and its complement 1023′), cellular barcode 1024 (and its complement 1024′), unique molecular identifier 1025 (and its complement 1025′), TSO sequence 1026 (and its complement 1026′), a poly-dG tract (and its complement a poly-dC tract 1046) (neither shown in this FIG. 1H-B), editing cassette transcript 1045 (and its complement 1045′), cassette capture sequence 1044 (and its complement 1044′), and priming sequence 1042 (and its complement 1042′). In processing of size-selected DNA duplex 1060, enzymatic fragmentation is not performed. Instead, two primers are added to size-selected duplex DNA 1060: 1) a P5 primer 1068 comprising a P5 sequence 1069 and a read 1 primer sequence 1070; and 2) primer sequence 1080 with a sequence 1084 complementary to priming sequence 1042 and a read 2 sequencing primer binding sequence 1067.

In a next step, sample indexing is performed using the P5 primer 1068 comprising a P5 sequence 1069 and a read 1 primer sequence 1070 used in the previous step with a P7/sample index primer 1071 comprising a P7 sequence 1072, a sample index 1073 and a read 2 primer 1074. Amplification with P5 primer 1068 and P7/sample index primer 1071 results in final DNA library constructs 1090 created from cellular editing cassettes ready for sequencing on any of the ILLUMINA sequencing systems. Final DNA library constructs 1090 comprise from 5′ to 3′ P5 sequence 1069 (and its complement 1069′), read 1 sequencing primer binding sequence 1023 (and its complement 1023′), cellular barcode 1024 (and its complement 1024′), unique molecular identifier 1025 (and its complement 1025′), TSO sequence 1026 (and its complement 1026′), editing cassette sequence 1045 (and its complement 1045′), cassette capture sequence 1044 (and its complement 1044′), priming sequence 1042 (and its complement 1042′), read 2 sequencing primer binding sequence 1067 (and its complement 1067′), sample index 1073 (and its complement 1073′), and P7 sequence 1072 (and its complement 1072′).

The exemplary processes in FIGS. 1D-1H depict converting random cellular nucleic acids and editing cassettes into constructs suitable for sequencing and correlating cellular nucleic acids and the edits that were made to the cell. FIG. 1I is a depiction of the processes of reverse transcription and template switching for mRNA transcripts (as opposed to random cellular nucleic acids generally) to create cDNAs in a cell after poly-dT primers and template switching oligonucleotides have been combined with the lysate of an individual cell (e.g., step 113 of FIG. 1A). At top left of FIG. 1I is a template switching oligonucleotide (TSO) 1022. TSO comprises from left (5′) to right (3′) a read 1 sequencing primer binding sequence 1023, a cellular barcode 1024, a unique molecular identifier 1025 and a TSO sequence 1026 comprising a poly-dG tract 1027. Cellular barcode 1024 is unique to each partition whether the partition is a droplet or a well, and the unique molecular identifiers 1025 comprise a tract of nucleotides coupled with a particular cellular barcode where each unique molecular identifier coupled with a particular cellular barcode is different. Cellular barcode 1024 facilitates association of cDNAs created from the mRNA and editing cassette transcripts originating from a single cell, and the unique molecular identifiers allow tracking of cDNAs originating from a single cDNA after amplification.

At top right of FIG. 1I is an mRNA transcript 1094 comprising a poly-A tract at the 3′ end. Below mRNA transcript 1093 is a poly-dT primer 1092 comprising a priming sequence 1029 and a poly-dT tract 1092 that can capture the poly-A tracts of mRNAs. In a next step, the mRNA transcript 1094 and poly-dT primer 1092 are hybridized and a copy is made 1094 of mRNA transcript 1094 resulting in a reverse transcript construct 1093. During reverse transcription of the mRNA transcript several to many untemplated Cs 1032 are added to the 3′ end of the reverse transcript construct 1093. Untemplated Cs 1032 are capable of hybridizing with the poly-dG tract 1027 of TSO 1022 (allowing for, e.g., TSO priming). After TSO priming of mRNA transcript 1094 and reverse transcript construct 1093, the reverse transcript construct 1093 is extended from the untemplated Cs 1032 to include TSO sequence complement 1026′, unique molecular identifier complement 1025′, cellular barcode complement 1024′, and read 1 sequencing primer binding sequence complement 1023′ resulting in an extended cDNA transcript 1036.

### Automated Cell Editing Instruments and Modules to Perform Nucleic Acid-Guided Nuclease or Nickase Fusion Editing in Cells

Automated Cell Editing Instruments

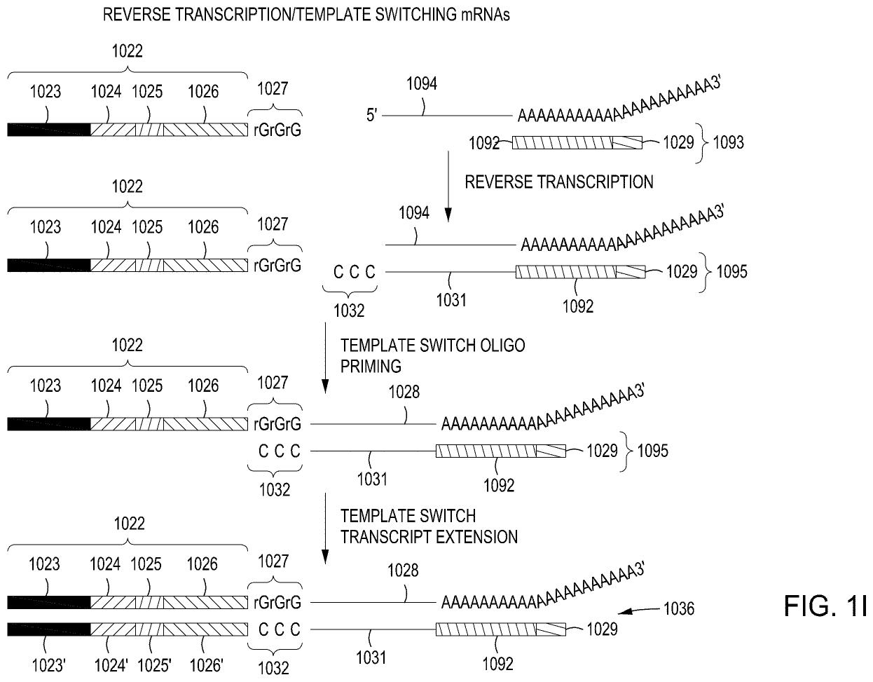

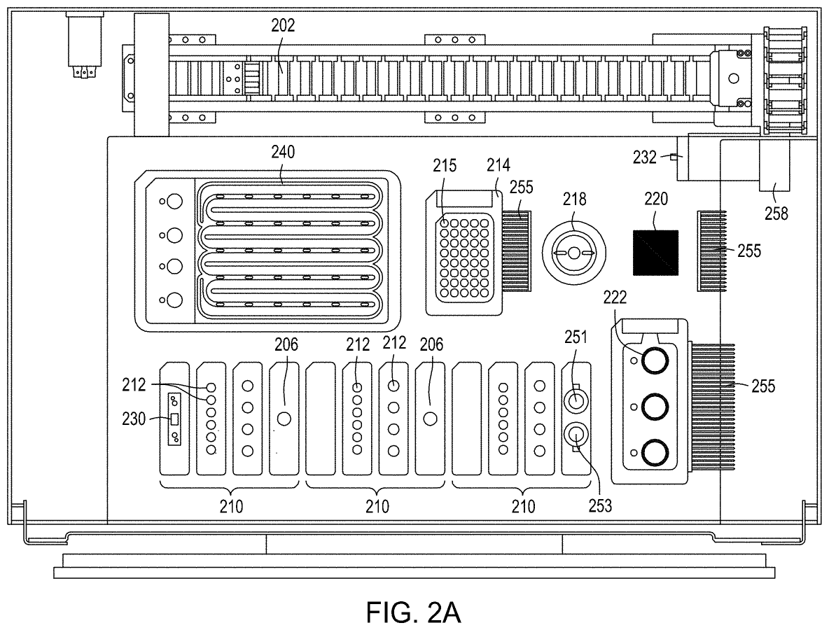

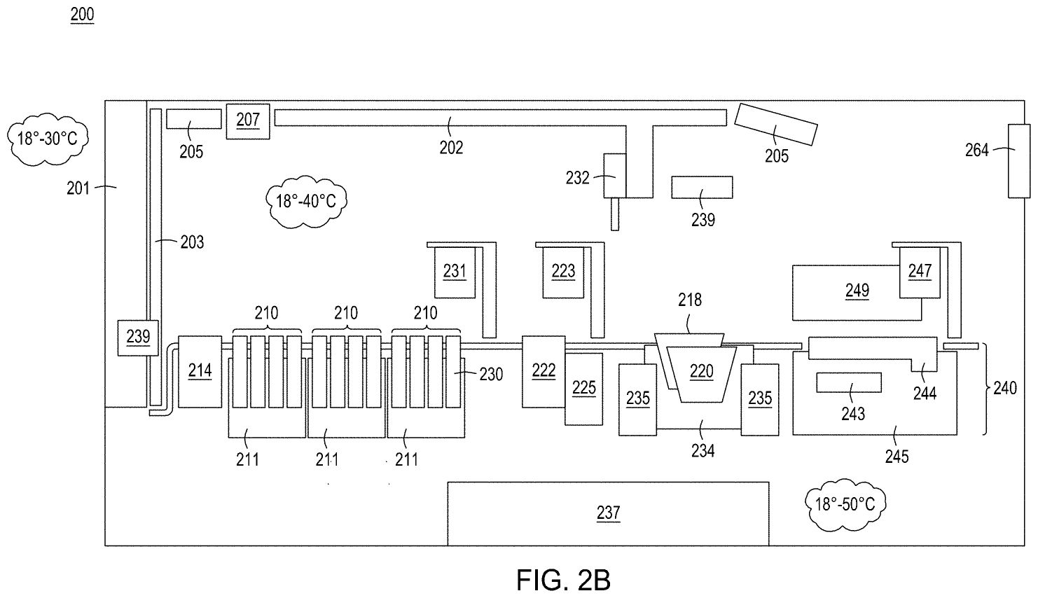

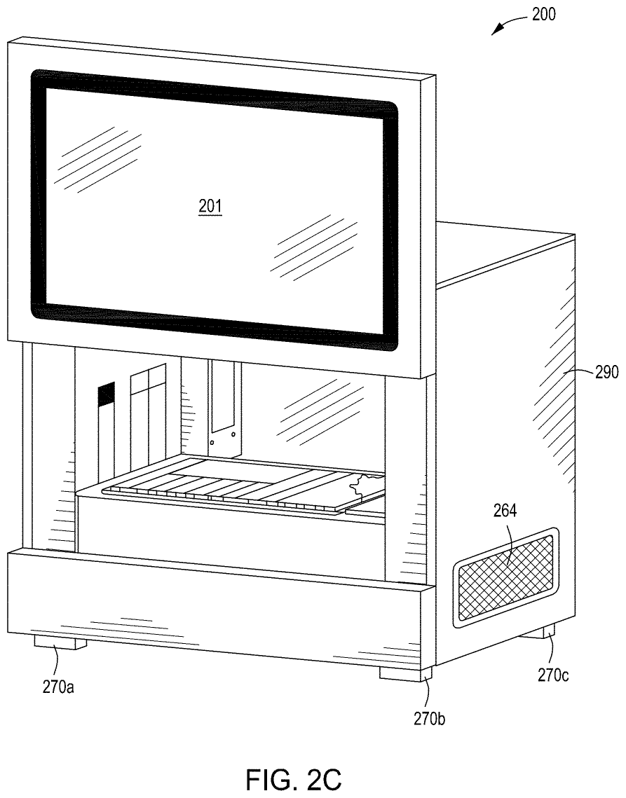

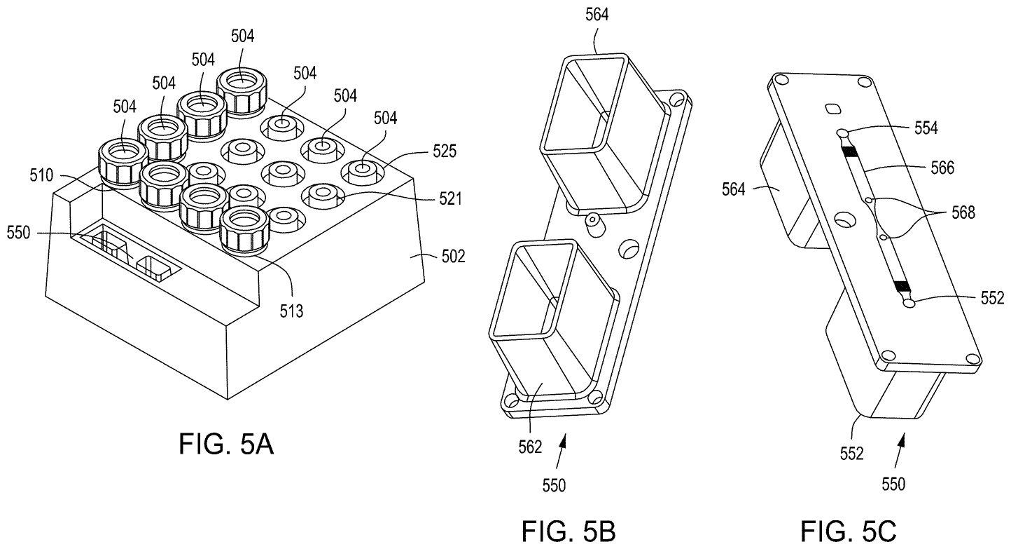

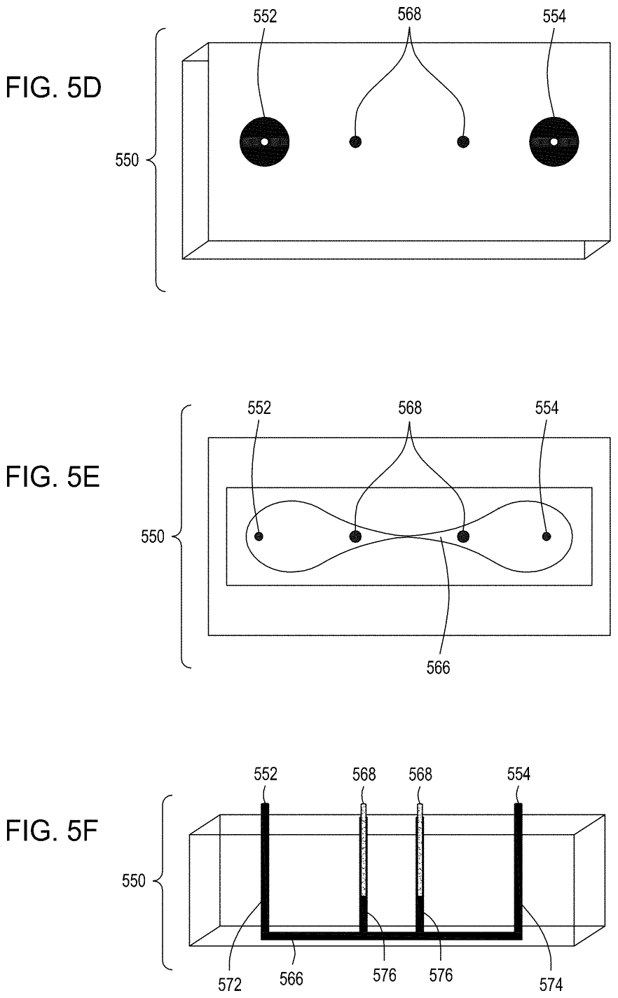

FIG. 2A depicts an exemplary automated multi-module cell processing instrument 200 to, e.g., perform targeted gene editing of live cells and for assessing the consequences of the edits. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 5B-5F), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash cartridge 204 may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 232. The robotic liquid handling system allows for the transfer of liquids between modules without human intervention.

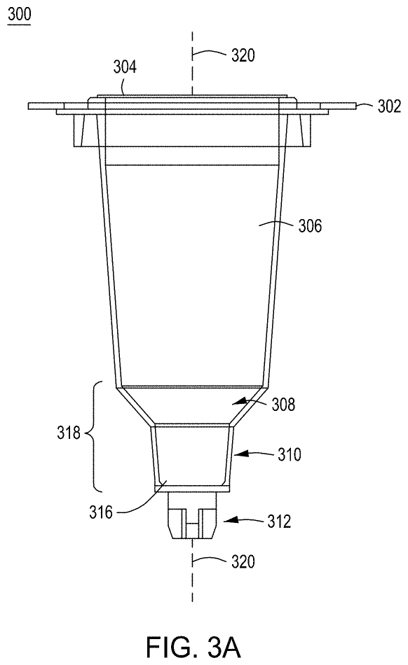

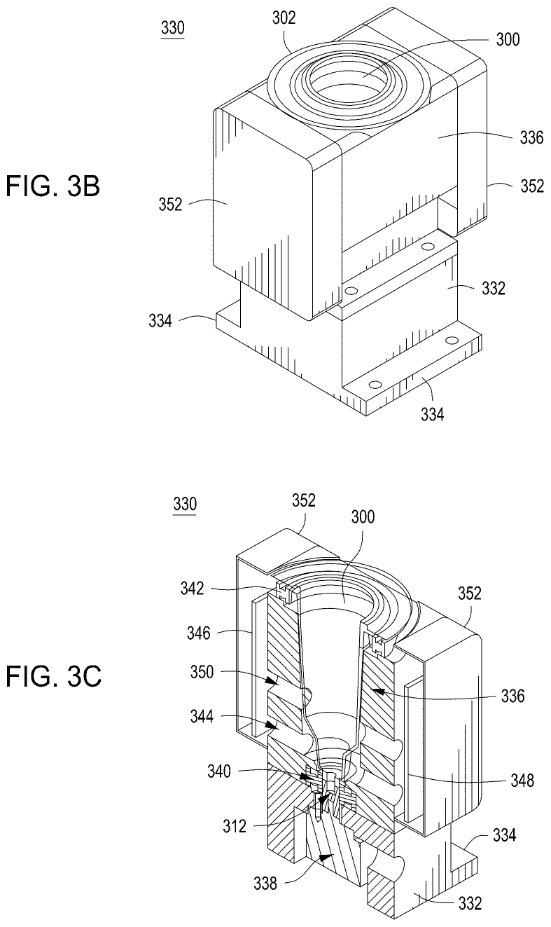

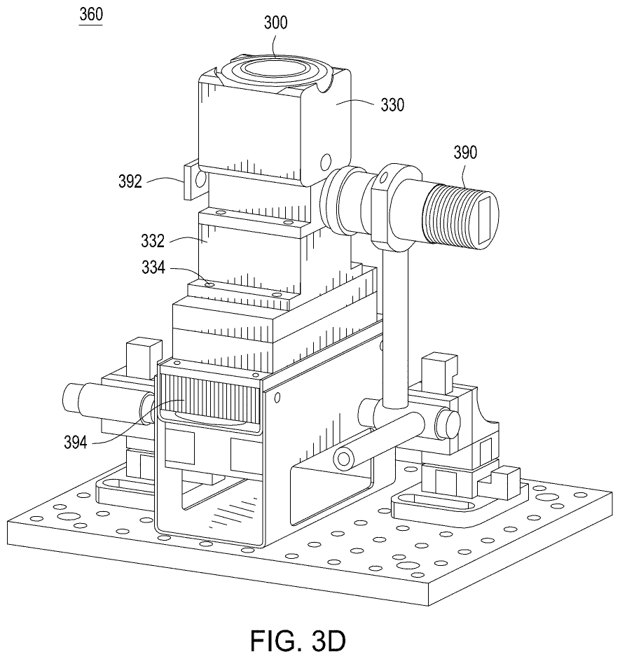

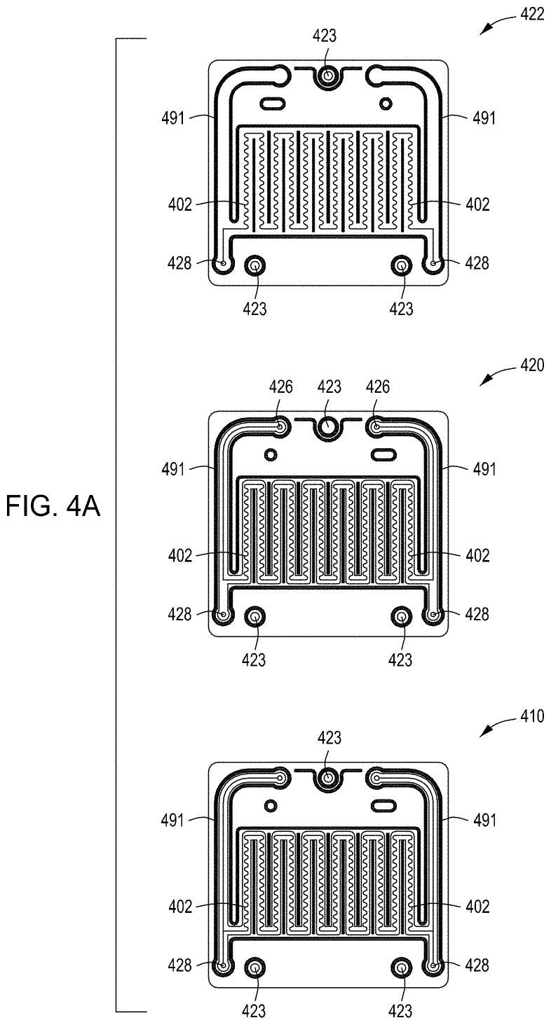

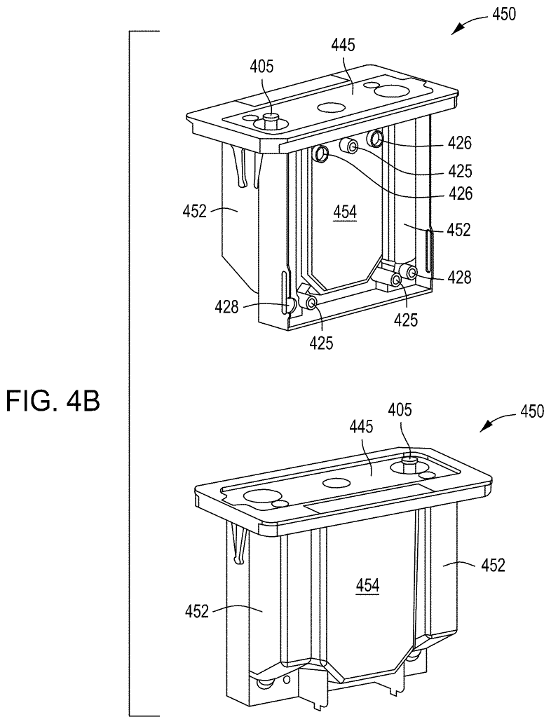

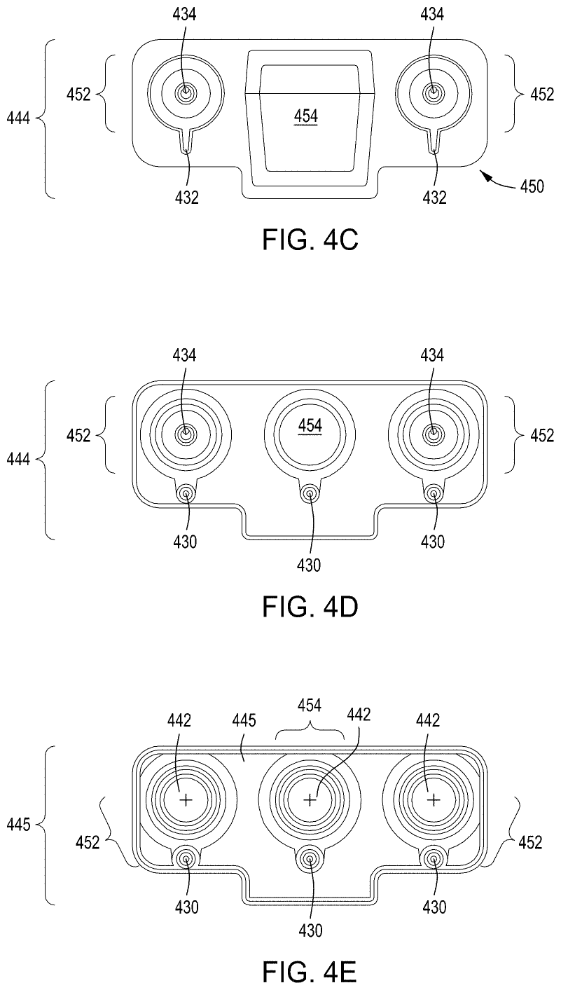

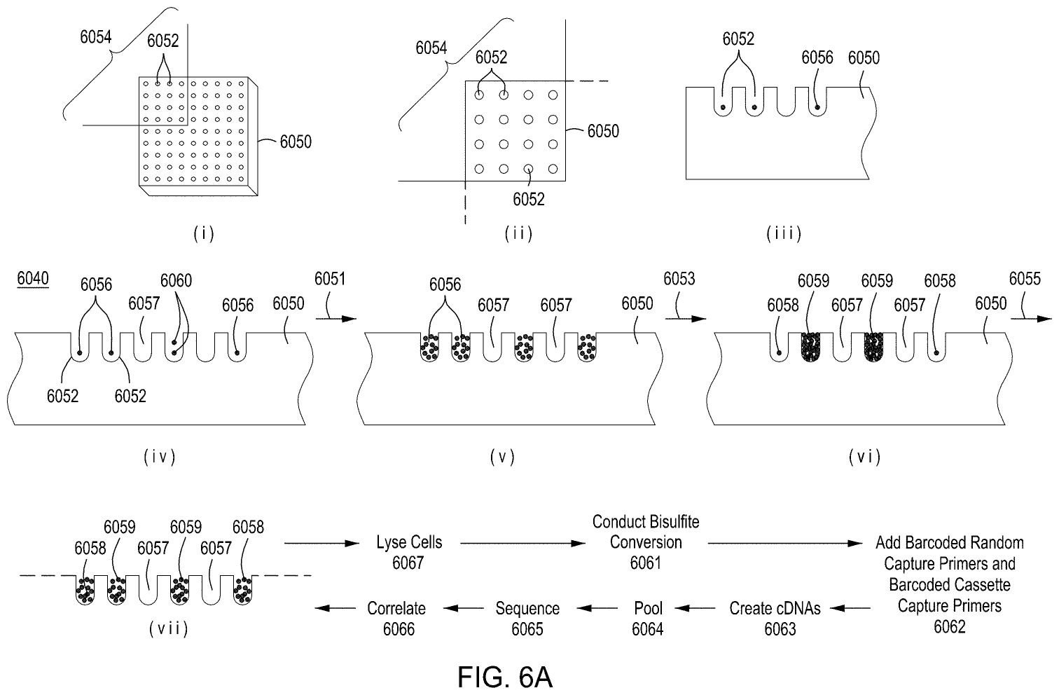

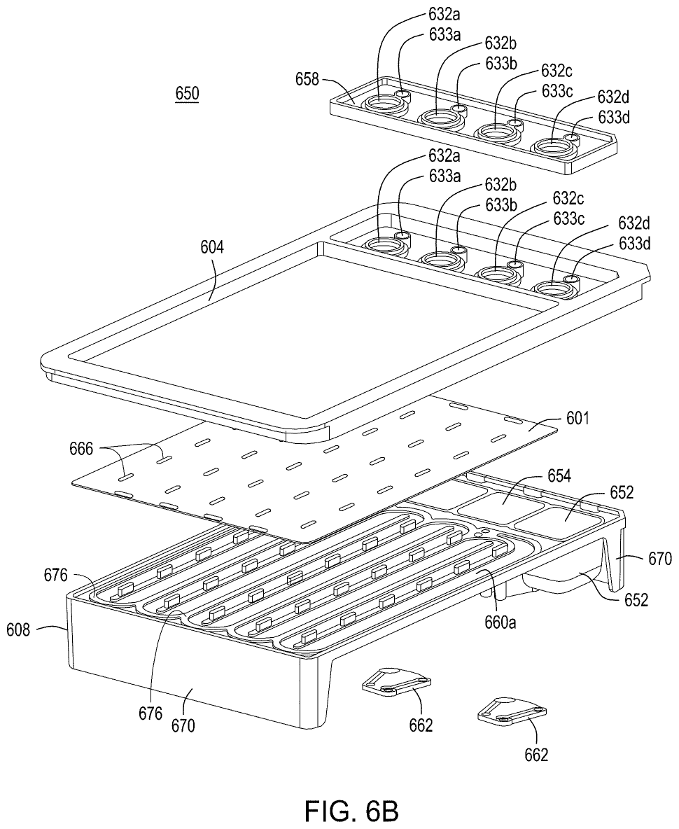

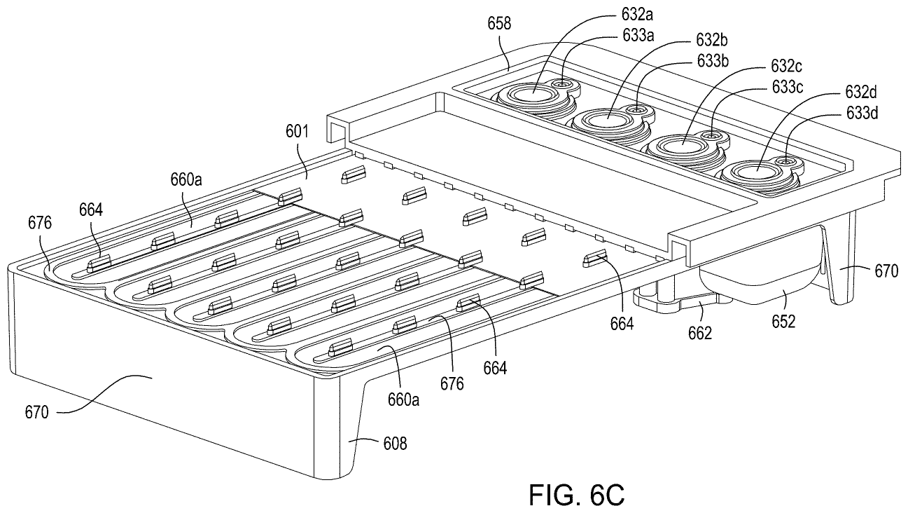

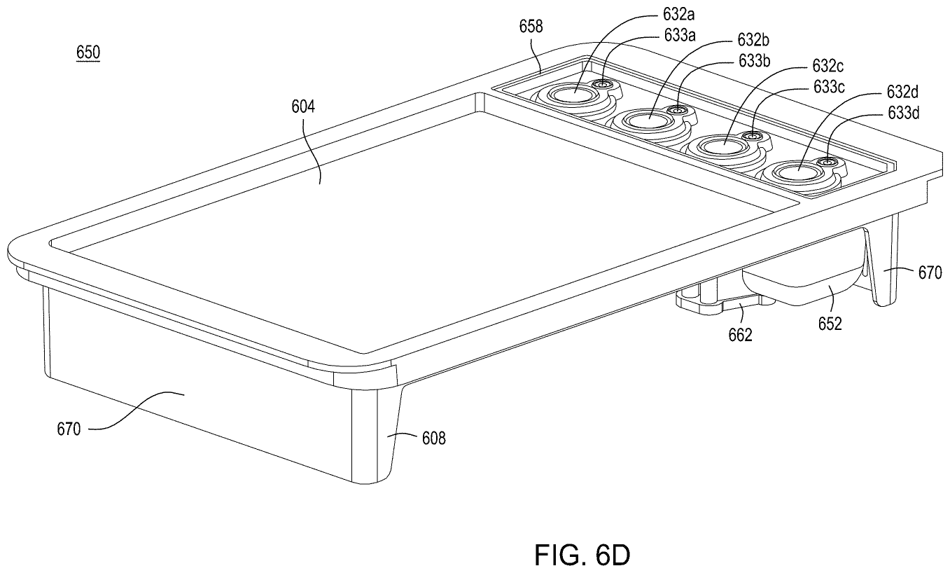

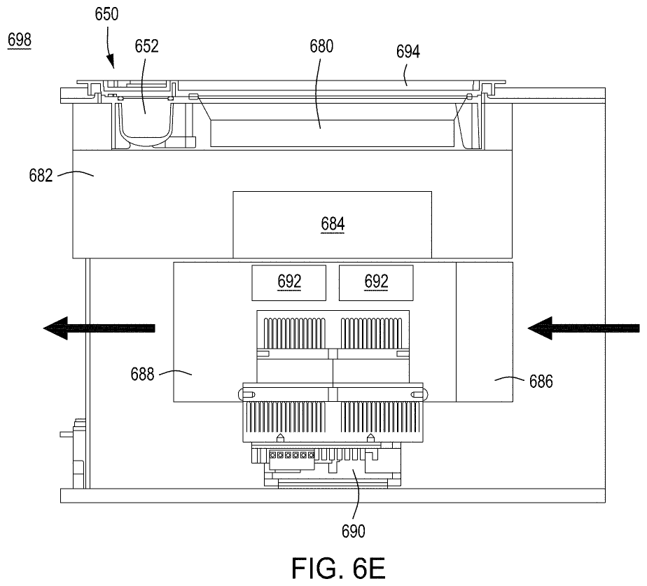

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (described in greater detail below in relation to FIGS. 3A-3D). Additionally seen is the TFF module 222 (described above in detail in relation to FIGS. 4A-4E). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6C-6F, served by, e.g., robotic liquid handing system 258 and air displacement pipettor 232. Additionally seen is a selection module 220. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. Selection module is seen at 220. Also seen is the SWIIN module 240, comprising a SWIIN cartridge 241, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIG. 2C illustrates a front perspective view of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019 and U.S. Ser. No. 16/412,195, filed 14 May 2019; Ser. No. 16/680,643, filed 12 Nov. 2019; and Ser. No. 16/750,369, filed 23 Jan. 2020, all of which are herein incorporated by reference in their entirety.

### The Rotating Cell Growth Module

FIG. 3A shows one embodiment of a rotating growth vial 300 for use with the cell growth device and in the automated multi-module cell processing instruments described herein for growing cells (e.g., bacterial, yeast or animal) in suspension. Bacterial and yeast cells are typically grown in suspension. Growing mammalian cells in suspension (e.g., even adherent cells) can be effected in various forms. Adherent cells that typically are grown in 2D cultures when grown in suspension often aggregate into “clumps.” For example, some mammalian cells grow well as aggregates in suspension, and are most healthy growing in aggregates of 50-300 microns in size, starting off as smaller aggregates 30-50 microns in size. Mammalian cells are typically grown in culture 3-5 days between passaging and the larger aggregates are broken into smaller aggregates by filtering them, e.g., through a cell strainer (e.g., a sieve) with a 37 micron filter. The mammalian cells can grow indefinitely in 3D aggregates as long as they are passaged into smaller aggregates when the aggregates become 300-400 microns in size.

An alternative to growing cells in 3D aggregates is growing cells on microcarriers. Generally, microcarriers are nonporous (comprised of pore sizes range from 0-20 nm), microporous (comprised of pore sizes range from 20 nm-1 micron), and macroporous (comprised of pore sizes range from 1-50 microns) microcarriers comprising natural organic materials such as, e.g., gelatin, collagen, alginate, agarose, chitosan, and cellulose, synthetic polymeric materials such as, e.g., polystyrene, polyacrylates such as polyacrylamide, polyamidoamine (PAMAM), polyethylene oxide (PEO/PEG), poly(N-isopropylacrylamide) (PNIPAM), polycaprolactone (PCL), polylactic acid (PLA), and polyglycolic acid (PGA), inorganic materials such as, e.g., silica, silicon, mica, quartz and silicone, as well as mixtures of natural, polymeric materials, cross-linked polymeric materials, and inorganic materials etc. on which animal cells can grow. Microcarriers useful for the methods herein typically range in size from 30-1200 microns in diameter and more typically range in size from 40-200 or from 50-150 microns in diameter.

Finally, another option for growing mammalian cells for editing in the compositions, methods, modules and automated instruments described herein is growing single cells in suspension using a specialized medium such as that developed by Accellta™ (Haifa, Israel). Cells grown in this medium must be adapted to this process over many cell passages; however, once adapted the cells can be grown to a density of >40 million cells/ml and expanded 50-100× in approximately a week, depending on cell type.

The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some embodiments of the rotating growth vial 300 have a second light path 308 in the tapered region of the tapered-to-constricted region 318. Both light paths in this embodiment are positioned in a region of the rotating growth vial that is constantly filled with the cell culture (cells+growth media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some embodiments, the motor drives the drive engagement mechanism 312 such that the rotating growth vial 300 is rotated in one direction only, and in other embodiments, the rotating growth vial 300 is rotated in a first direction for a first amount of time or periodicity, rotated in a second direction (i.e., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some embodiments, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 302 to overlap and engage with the cell growth device. In automated systems, the rotating growth vial 300 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 300 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 300. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 3B is a perspective view of one embodiment of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail. In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth via 316. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some embodiments, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some embodiments to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 330—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth device 330 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this embodiment, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 430 may include additional sensors for measuring, e.g., dissolved oxygen, carbon dioxide, pH, conductivity, and the like. For additional details regarding rotating growth vials and cell growth devices see U.S. Pat. No. 10,435,662, issued 8 Oct. 2019; U.S. Pat. No. 10,443,031, issued 15 Oct. 2019; U.S. Pat. No. 10,590,375, issued 17 Mar. 2020; and U.S. Ser. No. 16/780,640, filed 3 Feb. 2020; and Ser. No. 16/836,664, filed 31 Mar. 2020.

### The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in automated, integrated, multi-module instruments for the processes listed above is a module or component that can grow, perform buffer exchange, and/or concentrate cells and render them competent so that they may be transformed or transfected with the nucleic acids needed for engineering or editing the cell's genome.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422—specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this embodiment mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple and tangential flow assembly 410 to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this embodiment has a serpentine configuration and an undulating geometry, although other geometries may be used. Permeate member 420 also comprises countersinks 423, coincident with the countersinks 423 on retentate member 420.

On the left of FIG. 4A is a tangential flow assembly 410 comprising the retentate member 422 and permeate member 420 seen in this FIG. 4A. In this view, retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 102 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 422 and permeate 420 members may be different depending on the depth of the channel in each member.

FIG. 4B shows front perspective (right) and rear perspective (left) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition is seen gasket 445 covering the top of reservoir assembly 450. Gasket 445 is described in detail in relation to FIG. 4E. At left in FIG. 4B is a rear perspective view of reservoir assembly 1250, where “rear” is the side of reservoir assembly 450 that is not coupled to the tangential flow assembly. Seen are retentate reservoirs 452, permeate reservoir 454, and gasket 445.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIGS. 4B and 4E depicts a gasket 445 that in operation is disposed on cover 444 of reservoir assemblies 450 shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configures to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall workflow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports 406, collecting the cell culture through a second retentate port 404 into a second retentate reservoir, optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports 406. Alternatively, cells can be grown in one reservoir with bubbling or agitation without passing the cells through the TFF channel from one reservoir to the other.

The overall workflow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420) of the device. In this process, a fixed volume of cells in medium or buffer is driven through the device until the cell sample is collected into one of the retentate ports 404, and the medium/buffer that has passed through the membrane is collected through one or both of the permeate/filtrate ports 406. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells—except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen- or ECM- (extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports 404 while collecting the medium in one of the permeate/filtrate ports 406 is considered “one pass” of the cell sample. The transfer between retentate reservoirs “flips” the culture. The retentate and permeate ports collecting the cells and medium, respectively, for a given pass reside on the same end of TFF device/module with fluidic connections arranged so that there are two distinct flow layers for the retentate and permeate/filtrate sides, but if the retentate port 404 resides on the retentate member of device/module (that is, the cells are driven through the channel above the membrane and the filtrate (medium) passes to the portion of the channel below the membrane), the permeate/filtrate port 406 will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port 404 and into the retentate reservoir (not shown). To initiate another “pass”, the cell sample is passed again through the TFF device, this time in a flow direction that is reversed from the first pass. The cell sample is collected by passing through the retentate port 404 and into retentate reservoir (not shown) on the opposite end of the device/module from the retentate port 404 that was used to collect cells during the first pass. Likewise, the medium/buffer that passes through the membrane on the second pass is collected through the permeate port 406 on the opposite end of the device/module from the permeate port 406 that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously. For further information and alternative embodiments on TFFs see, e.g., U.S. Ser. No. 16/798,302, filed 22 Feb. 2020.

### The Cell Transformation Module

FIG. 5A depicts an exemplary combination reagent cartridge and electroporation device 500 (“cartridge”) that may be used in an automated multi-module cell processing instrument along with the TFF module. In addition, in certain embodiments the material used to fabricate the cartridge is thermally-conductive, as in certain embodiments the cartridge 500 contacts a thermal device (not shown), such as a Peltier device or thermoelectric cooler, that heats or cools reagents in the reagent reservoirs or reservoirs 504. Reagent reservoirs or reservoirs 504 may be reservoirs into which individual tubes of reagents are inserted as shown in FIG. 5A, or the reagent reservoirs may hold the reagents without inserted tubes. Additionally, the reservoirs in a reagent cartridge may be configured for any combination of tubes, co-joined tubes, and direct-fill of reagents.

In one embodiment, the reagent reservoirs or reservoirs 504 of reagent cartridge 500 are configured to hold various size tubes, including, e.g., 250 ml tubes, 25 ml tubes, 10 ml tubes, 5 ml tubes, and Eppendorf or microcentrifuge tubes. In yet another embodiment, all reservoirs may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one of ordinary skill in the art will appreciate given the present disclosure, the reagents contained in the reagent cartridge will vary depending on workflow; that is, the reagents will vary depending on the processes to which the cells are subjected in the automated multi-module cell processing instrument, e.g., protein production, cell transformation and culture, cell editing, etc.

Reagents such as cell samples, enzymes, buffers, nucleic acid vectors, expression cassettes, proteins or peptides, reaction components (such as, e.g., MgCl₂, dNTPs, nucleic acid assembly reagents, gap repair reagents, medium and the like), wash solutions, ethanol, and magnetic beads for nucleic acid purification and isolation, etc. may be positioned in the reagent cartridge at a known position. In some embodiments of cartridge 500, the cartridge comprises a script (not shown) readable by a processor (not shown) for dispensing the reagents. Also, the cartridge 500 as one component in an automated multi-module cell processing instrument may comprise a script specifying two, three, four, five, ten or more processes to be performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components/modules of the automated multi-module cell processing instrument or system may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument, or, e.g., reagents for protein expression and a script that specifies the process steps for performing protein expression in an automated multi-module cell processing instrument.

For example, the reagent cartridge may comprise a script to pipette competent cells from a reservoir, transfer the cells to a transformation module, pipette a nucleic acid solution comprising a vector with expression cassette from another reservoir in the reagent cartridge, transfer the nucleic acid solution to the transformation module, initiate the transformation process for a specified time, then move the transformed cells to yet another reservoir in the reagent cassette or to another module such as a cell growth module in the automated multi-module cell processing instrument. In another example, the reagent cartridge may comprise a script to transfer a nucleic acid solution comprising a vector from a reservoir in the reagent cassette, nucleic acid solution comprising editing oligonucleotide cassettes in a reservoir in the reagent cassette, and a nucleic acid assembly mix from another reservoir to the nucleic acid assembly/desalting module, if present. The script may also specify process steps performed by other modules in the automated multi-module cell processing instrument. For example, the script may specify that the nucleic acid assembly/desalting reservoir be heated to 50° C. for 30 min to generate an assembled product; and desalting and resuspension of the assembled product via magnetic bead-based nucleic acid purification involving a series of pipette transfers and mixing of magnetic beads, ethanol wash, and buffer.

As described in relation to FIGS. 5B and 5C below, the exemplary reagent cartridges for use in the automated multi-module cell processing instruments may include one or more electroporation devices, preferably flow-through electroporation (FTEP) devices. In yet other embodiments, the reagent cartridge is separate from the transformation module. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archaea, yeasts, other eukaryotic cells, bacteria, and other cell types. Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif.) makes the GENE PULSER XCELL™ line of products to electroporate cells in cuvettes. Traditionally, electroporation requires high field strength; however, the flow-through electroporation devices included in the reagent cartridges achieve high efficiency cell electroporation with low toxicity. The reagent cartridges of the disclosure allow for particularly easy integration with robotic liquid handling instrumentation that is typically used in automated instruments and systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

FIGS. 5B and 5C are top perspective and bottom perspective views, respectively, of an exemplary FTEP device 550 that may be part of (e.g., a component in) reagent cartridge 500 in FIG. 5A or may be a stand-alone module; that is, not a part of a reagent cartridge or other module. FIG. 5B depicts an FTEP device 550. The FTEP device 550 has wells that define cell sample inlets 552 and cell sample outlets 554. FIG. 5C is a bottom perspective view of the FTEP device 550 of FIG. 5B. An inlet well 552 and an outlet well 554 can be seen in this view. Also seen in FIG. 5C are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Pat. No. 10,435,713, issued 8 Oct. 2019; U.S. Pat. No. 10,443,074, issued 15 Oct. 2019; U.S. Pat. No. 10,323,258, issued 18 Jun. 2019; U.S. Pat. No. 10,568,288, issued 17 Dec. 2019; and U.S. Pat. No. 10,415,058, issued 17 Sep. 2019. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889, issued 13 Aug. 2019; U.S. Pat. No. 10,406,525, issued 10 Sep. 2019; U.S. Pat. No. 10,478,822, issued 19 Nov. 2019; and U.S. Ser. No. 16/596,940, filed 9 Oct. 2019.

Additional details of the FTEP devices are illustrated in FIGS. 5D-5F. Note that in the FTEP devices in FIGS. 5D-5F the electrodes are placed such that a first electrode is placed between an inlet and a narrowed region of the flow channel, and the second electrode is placed between the narrowed region of the flow channel and an outlet. FIG. 5D shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device. FIG. 5E shows a cutaway view from the top of the FTEP device 550, with the inlet 552, outlet 554, and electrodes 568 positioned with respect to a flow channel 566. FIG. 5F shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 576 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining. The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 508 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired-as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the invention, the flow-through electroporation device comprises at least one additional electrode which applies a ground potential to the FTEP device. Flow-through electroporation devices (either as a stand-alone instrument or as a module in an automated multi-module system) are described in, e.g., U.S. Pat. No. 10,435,713, issued 8 Oct. 2019; U.S. Pat. No. 10,443,074, issued 15 Oct. 2019; U.S. Pat. No. 10,323,258, issued 30 Sep. 2019; U.S. Pat. No. 10,508,288, issued 17 Dec. 2019; U.S. Pat. No. 10,415,058, issued 17 Sep. 2019; and U.S. Pat. No. 10,557,150, issued 11 Feb. 2020; and U.S. Ser. No. 16/550,790, filed 26 Aug. 2019; and Ser. No. 16/548,208, filed 22 Aug. 2019.

### Cell Singulation and Enrichment Device

FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, Poisson or substantial Poisson loading has taken place; that is, each microwell has one or no cells, and the likelihood that any one microwell has more than one cell is low. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then editing is allowed to occur 6053. For growth and editing, the medium used will depend, of course, on the type of cells being edited—e.g., bacterial, yeast or mammalian. For example, medium for mammalian cell growth includes MEM, DMEM, IMDM, RPMI, Hanks', PBS and Ringer's solution.

After editing 6053, many cells in the colonies of cells that have been edited die as a result of the double-strand cuts caused by active editing and there is a lag in growth for the edited cells that do survive but must repair and recover following editing (microwells 6058), where cells that do not undergo editing thrive (microwells 6059) (vi). All cells are allowed to continue grow to establish colonies and normalize, where the colonies of edited cells in microwells 6058 catch up in size and/or cell number with the cells in microwells 6059 that do not undergo editing (vii). Once the cell colonies are normalized, the cells are lysed 6067, the nucleic acids are treated to bisulfite conversion 6061 as described supra, barcoded random capture primers and barcoded cassette capture primers are added to the cell lysate 6062, cDNAs (or DNA copies) are made 6063, and the barcoded cellular nucleic acids are pooled 6064, sequenced 6065, and the cellular nucleic acids are correlated 6066 to the edit(s) made.

A module useful for performing the methods depicted in FIG. 6A is a solid wall isolation, incubation, and normalization (SWIIN) module. FIG. 6B depicts an embodiment of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6B comprises from the top down, a reservoir gasket or cover 658, a retentate member 604 (where a retentate flow channel cannot be seen in this FIG. 6C), a perforated member 601 swaged with a filter (filter not seen in FIG. 6B), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 can be seen disposed on the top of permeate member 608 as well. The perforations that form the wells on perforated member 601 are not seen in this FIG. 6B; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6B).

In this FIG. 6B, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nL, with a total of approximately 200,000 microwells. The distance between the microwells is approximately 279 μm center-to-center. Though here the microwells have a volume of approximately 2.5 nL, the volume of the microwells may be from 1 to 25 nL, or preferably from 2 to 10 nL, and even more preferably from 2 to 4 nL. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 6E and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLOS ONE, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See, e.g., FIG. 6E and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through permeate ports 611. The medium flows upward through filter 603 to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter 603 and pooled. Alternatively, if cherry picking is desired, the growth of the cell colonies in the microwells is monitored, and slow-growing colonies are directly selected; or, fast-growing colonies are eliminated.

FIG. 6C is a top perspective view of a SWIIN module with the retentate and perforated members in partial cross section. In this FIG. 6C, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6C) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6C) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes 666 of perforated member 601. There is also a support 670 at the end distal reservoirs 652, 654 of permeate member 608.

FIG. 6D is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight).

After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6E depicts the embodiment of the SWIIN module in FIGS. 6B-6E further comprising a heat management system including a heater and a heated cover. The heater cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6F, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see U.S. Pat. No. 10,533,152, issued 14 Jan. 2020; U.S. Pat. No. 10,532,324, issued 14 Jan. 2020; U.S. Pat. No. 10,550,363, issued 4 Feb. 2020; U.S. Pat. No. 10,625,212, issued 21 Apr. 2020; U.S. Pat. No. 10,663,626, issued 28 Apr. 2020; U.S. Pat. No. 10,633,627, issued 28 Apr. 2020; U.S. Pat. No. 10,647,958, issued 12 May 2020; and U.S. Ser. No. 16/823,269, filed 18 Mar. 2020; Ser. No. 16/820,292, filed 16 Mar. 2020; Ser. No. 16/844,339, filed 9 Apr. 2020; Ser. No. 16/686,066, filed 15 Nov. 2019; and Ser. No. 16/820,324, filed 16 Mar. 2020.

It should be apparent to one of ordinary skill in the art given the present disclosure that the processes described may be recursive and multiplexed; that is, cells may go through the workflow described in relation to FIG. 1A, then the resulting edited population of cells may go through another (or several or many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells. Curing can be accomplished by, e.g., cleaving the vector(s) using a curing plasmid thereby rendering the editing and/or engine vector (or single, combined vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine plasmid.

### Bioreactor

In addition to the rotating growth vial module shown in FIGS. 3A-3E and described in the related text, and the tangential flow filtration (TFF) module shown FIG. 4A-4G and described in the related text, a bioreactor can be used to grow cells off-instrument or to allow for cell growth and recovery on-instrument; e.g., as one module of the multi-module fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO 2019/046766; 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, Colo.) and Sartorius GmbH (Gottingen, Germany).

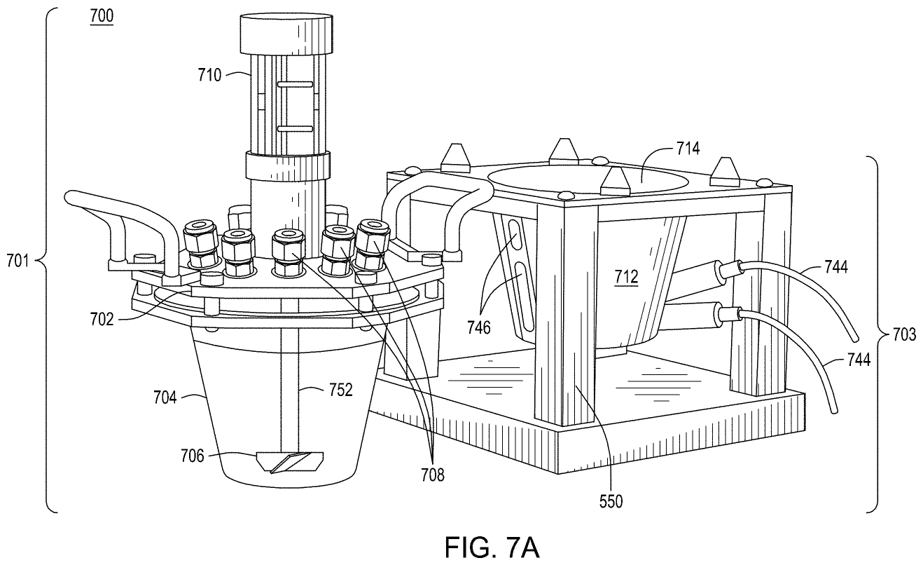

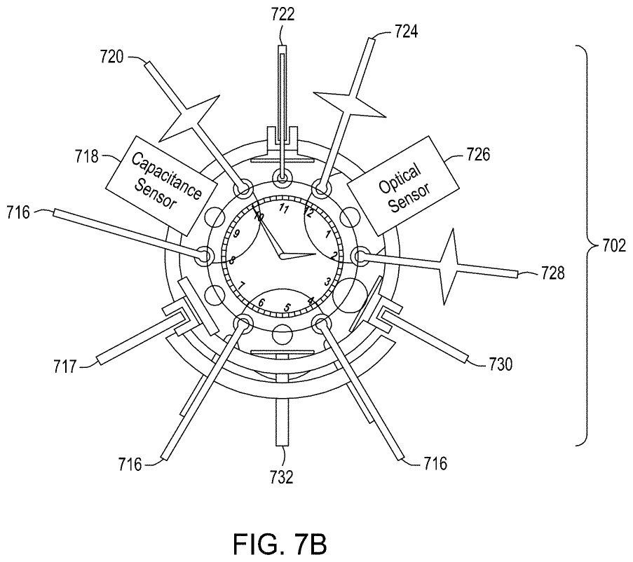

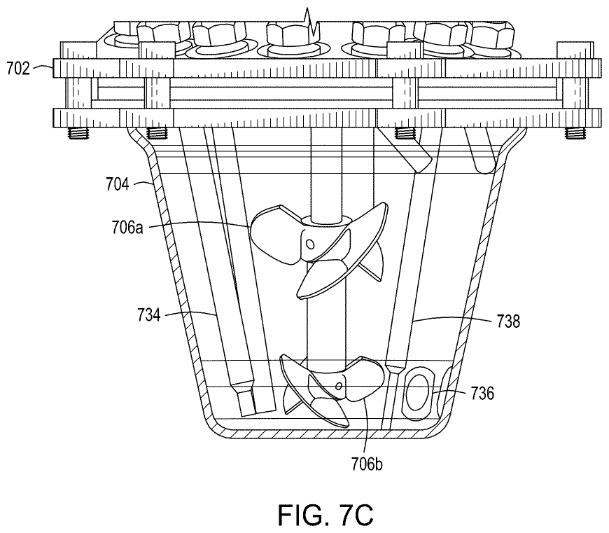

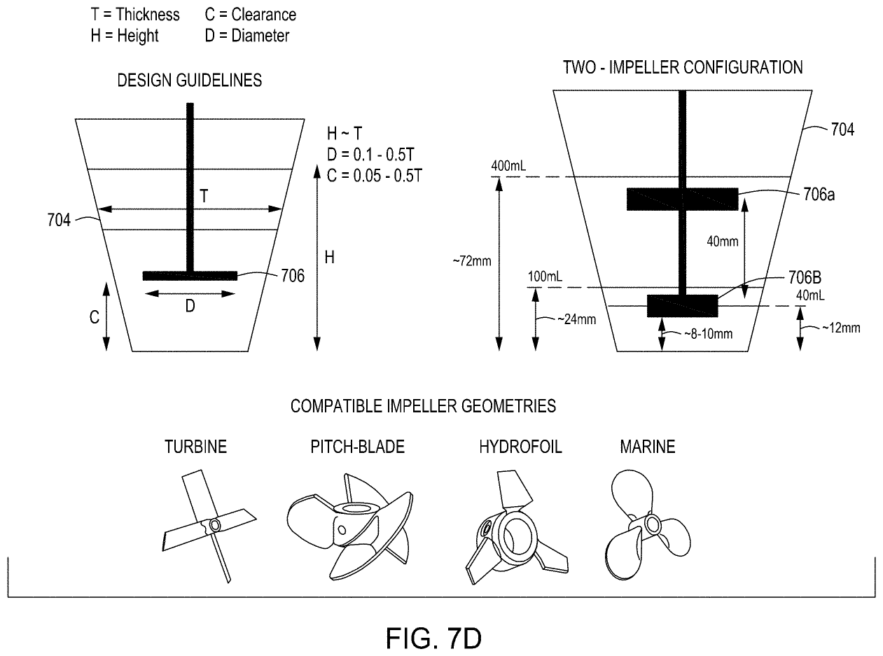

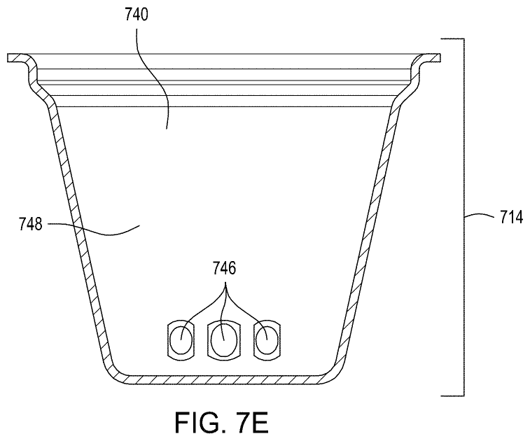

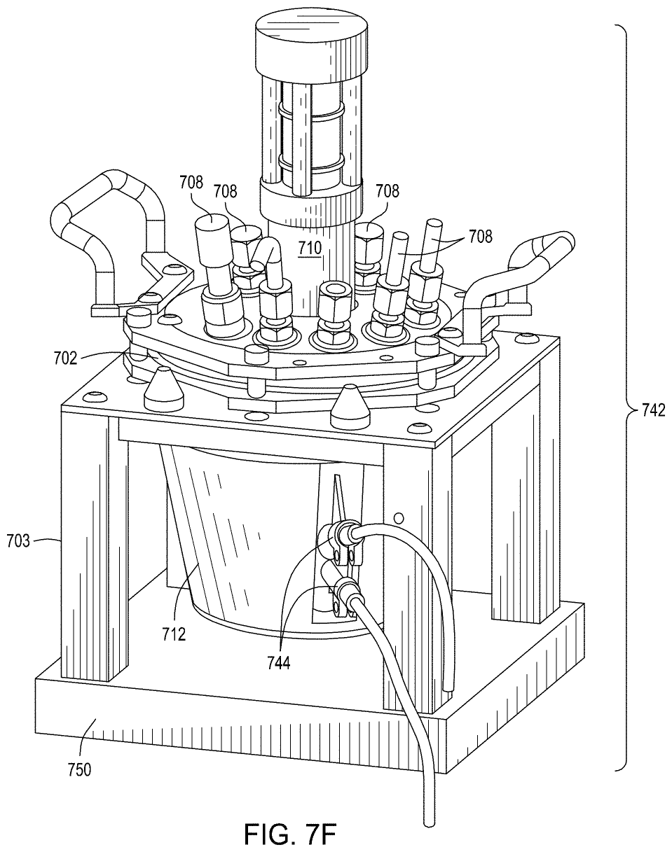

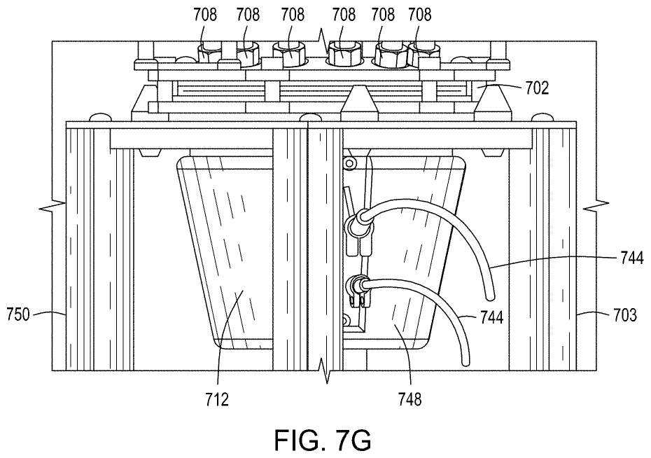

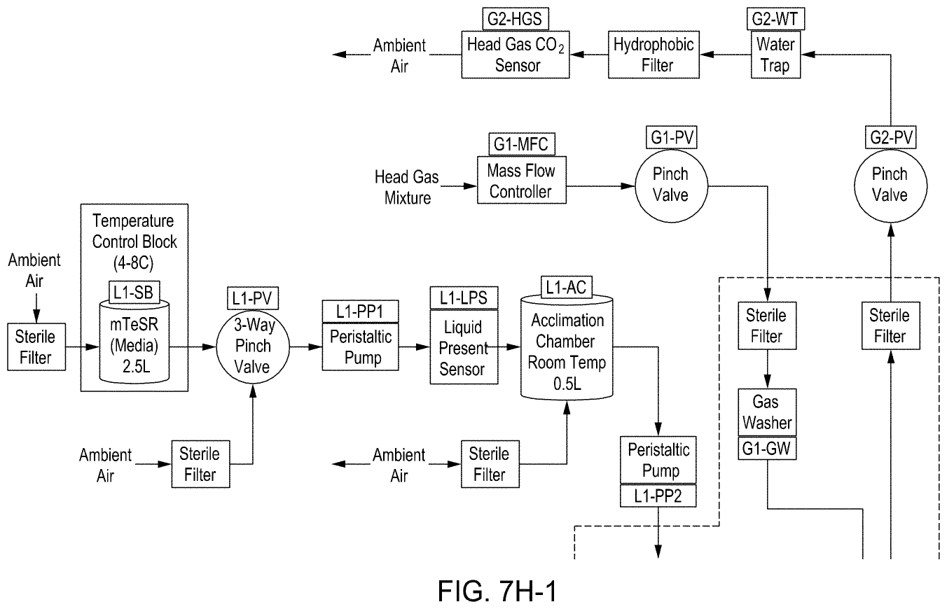

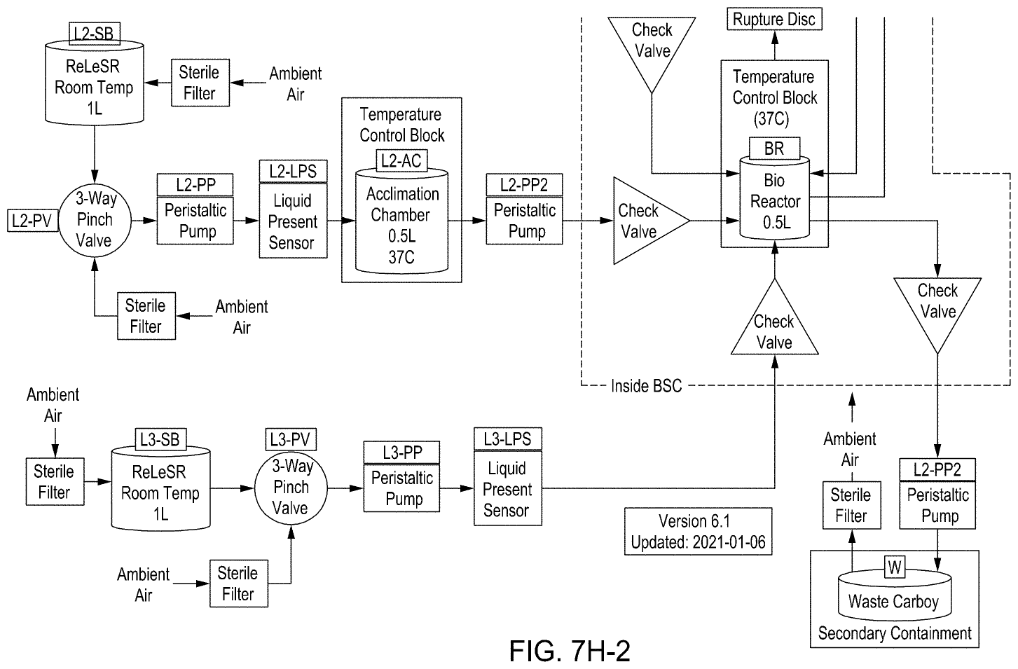

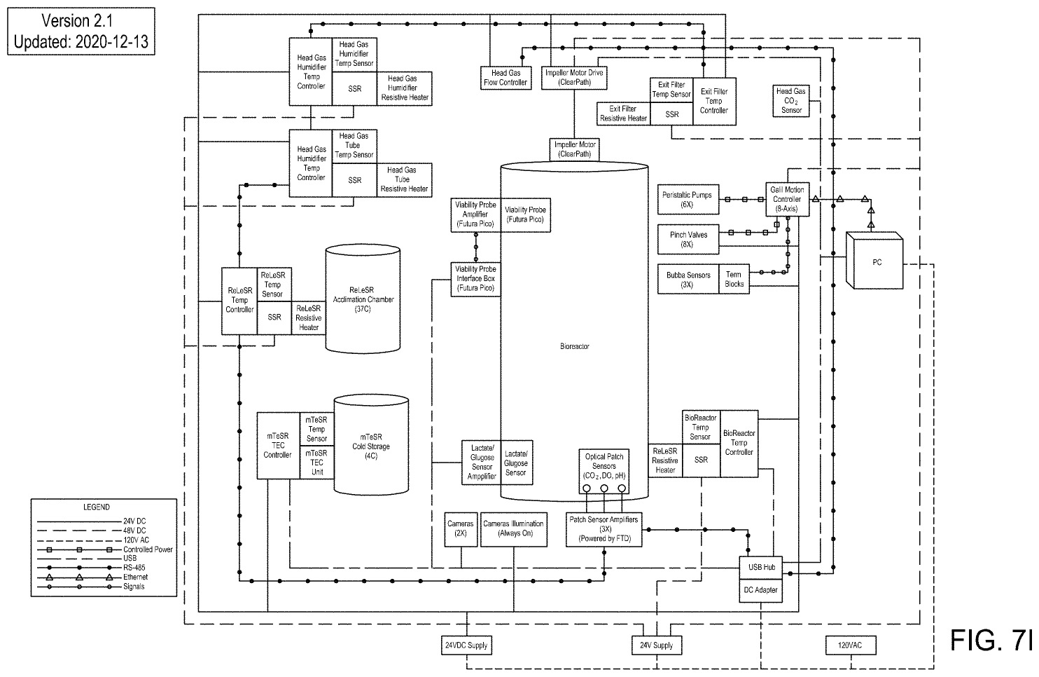

FIG. 7A shows one embodiment of a bioreactor assembly 700 for cell growth, transfection, and editing in the automated multi-module cell processing instruments described herein. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 700 comprises cell growth, transfection, and editing vessel 701 comprising a main body 704 with a lid assembly 702 comprising ports 708, including an optional motor integration port 710 driving impeller 706 via impeller shaft 752. Bioreactor assembly 700 comprises a growth vessel 701 comprising tapered a main body 704 with a lid assembly 702 comprising ports 708, including an optional motor integration port 710 driving impeller 706 via impeller shaft 752. The tapered shape of main body 704 of the vessel 701 along with, in some embodiments, dual impellers allows for working with a larger dynamic range of volumes, such as, e.g., up to 700 ml and as low as 100 ml for rapid sedimentation of the microcarriers. In addition, the low volume is useful for magnetic bead separation or enrichment as described above.

Bioreactor assembly 700 further comprises bioreactor stand assembly 703 comprising a main body 712 and vessel holder 714 comprising a heat jacket or other heating means (not shown, but see FIG. 7E) into which the main body 704 of vessel 701 is disposed in operation. The main body 704 of vessel 701 is biocompatible and preferably transparent—in some embodiments, in the UV and IR range as well as the visible spectrum—so that the growing cells can be visualized by, e.g., cameras or sensors integrated into lid assembly 702 or through viewing apertures or slots in the main body 712 of bioreactor stand assembly 703 (not shown in this FIG. 7A, but see FIG. 7E).

Bioreactor assembly 700 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 704 of vessel 701, the medium used to grow the cells, whether the cells are adherent or non-adherent. The bioreactor that comprises assembly 700 supports growth of both adherent and non-adherent cells, wherein adherent cells are typically grown of microcarriers as described in detail above and supra or as spheroids. Alternatively, another option for growing mammalian cells in the bioreactor described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/ml and expanded 50-100× in approximately a week, depending on cell type.

Main body 704 of vessel 701 preferably is manufactured by injection molding, as is, in some embodiments, impeller 706 and the impeller shaft (not shown). Impeller 706 also may be fabricated from stainless steel, metal, plastics or the polymers listed infra. Injection molding allows for flexibility in size and configuration and also allows for, e.g., volume markings to be added to the main body 704 of vessel 701. Additionally, material from which the main body 704 of vessel 701 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 704 of vessel 701 include those described for the rotating growth vial described in relation to FIGS. 3A and 3B and the TFF device described in relation to FIG. 4A-4E, including cyclic olefin copolymer (COC), glass, polyvinyl chloride, polyethylene, polyetheretherketone (PEEK), polypropylene, polycarbonate, poly(methyl methacrylate (PMMA)), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and is conducive to growth of both adherent and non-adherent cells and workflows involving microcarrier-based transfection. The main body 704 of vessel 701 may be reusable or, alternatively, may be manufactured and configured for a single use. In one embodiment, main body 704 of vessel 701 may support cell culture volumes of 25 ml to 500 ml, but may be scaled up to support cell culture volumes of up to 3 L.

The bioreactor stand assembly comprises a stand or frame 750, a main body 712 which holds the vessel 701 during operation. The stand/frame 750 and main body 712 are fabricated from stainless steel, other metals, or polymer/plastics. The bioreactor main body further comprises a heat jacket (not seen in FIG. 7A, but see FIG. 7E) to maintain the bioreactor main body 704—and thus the cell culture—at a desired temperature. Essentially, the stand assembly can host a set of sensors and cameras to monitor cell culture.

FIG. 7B depicts a top-down view of one embodiment of vessel lid assembly 702. Vessel lid assembly 702 is configured to be air-tight, providing a sealed, sterile environment for cell growth, transfection and editing as well as to provide biosafety maintaining a closed system. Vessel lid assembly 702 and the main body 704 of vessel 701 can be sealed via fasteners such as screws, using biocompatible glues, or the two components may be ultrasonically welded. Vessel lid assembly 702 is some embodiments is fabricated from stainless steel such as S316L stainless steel but may also be fabricated from metals, other polymers (such as those listed supra) or plastics. As seen in this FIG. 7B—as well as in FIG. 7A—vessel lid assembly 702 comprises a number of different ports to accommodate liquid addition and removal; gas addition and removal; for insertion of sensors to monitor culture parameters (described in more detail infra); to accommodate one or more cameras or other optical sensors; to provide access to the main body 704 of vessel 701 by, e.g., a liquid handling device; and to accommodate a motor for motor integration to drive one or more impellers 706. Exemplary ports depicted in FIG. 7B include three liquid-in ports 716 (at 4 o'clock, 6 o'clock and 8 o'clock), one liquid-out port 722 (at 11 'clock), a capacitance sensor 718 (at 9 o'clock), one “gas in” port 724 (at 12 o'clock), one “gas out” port 720 (at 10 o'clock), an optical sensor 726 (at 1 o'clock), a rupture disc 728 (at 2 o'clock), a self-sealing port 730 (at 3 o'clock) to provide access to the main body 704 of growth vessel 701; and a temperature probe 732 (at 5 o'clock).

The ports shown in vessel lid assembly 702 in this FIG. 7B are exemplary only and it should be apparent to one of ordinary skill in the art given the present disclosure that, e.g., a single liquid-in port 716 could be used to accommodate addition of all liquids to the cell culture rather than having a liquid-in port for each different liquid added to the cell culture. Similarly, there may be more than one gas-in port 724, such as one for each gas, e.g., O₂, CO₂ that may be added. In addition, although a temperature probe 732 is shown, a temperature probe alternatively may be located on the outside of vessel holder 714 of bioreactor stand assembly 503 separate from or integrated into heater jacket 748 (not seen in this FIG. 7B, but see FIG. 7E). A self-sealing port 730, if present, allows access to the main body 704 of vessel 701 for, e.g., a pipette, syringe, or other liquid delivery system via a gantry (not shown). As shown in FIG. 7A, additionally there may be a motor integration port to drive the impeller(s), although in other configurations of vessel 701 may alternatively integrate the motor drive at the bottom of the main body 704 of vessel 701. Vessel lid assembly 702 may also comprise a camera port for viewing and monitoring the cells.

Additional sensors include those that detect O₂ concentration, a CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk Conn.); PreSens Precision Sensing (Regensburg, Germany); C-CIT Sensors AG (Waedenswil, Switzerland), and ABER Instruments Ltd. (Alexandria, Va.). In one embodiment, optical density is measured using a reflective optical density sensor to facilitate sterilization, improve dynamic range and simplify mechanical assembly. The rupture disc, if present, provides safety in a pressurized environment, and is programmed to rupture if a threshold pressure is exceeded in the bioreactor. If the cell culture in the bioreactor vessel is a culture of adherent cells, microcarriers may be used as described supra. In such an instance, the liquid-out port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. The microcarriers used for initial cell growth can be nanoporous (where pore sizes are typically <20 nm in size), microporous (with pores between >20 nm to <1 μm in size), or macroporous (with pores between >1 μm in size, e.g. 20 μm) and the microcarriers are typically 50-200 μm in diameter; thus the pore size of the filter or frit in the liquid-out port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, Mo., USA); ThermoFisher Scientific (Waltham, Mass., USA); Pall Corp. (Port Washington, N.Y., USA); GE Life Sciences (Marlborough, Mass., USA); and Corning Life Sciences (Tewkesbury, Mass., USA). As for the extracellular matrix, natural matrices include collagen, fibrin and vitronectin (available, e.g., from ESBio, Alameda, Calif., USA), and synthetic matrices include MATRIGEL® (Corning Life Sciences, Tewkesbury, Mass., USA), GELTREX™ (ThermoFisher Scientific, Waltham, Mass., USA), CULTREX® (Trevigen, Gaithersburg, Md., USA), biomemetic hydrogels available from Cellendes (Tubingen, Germany); and tissue-specific extracellular matrices available from Xylyx (Brooklyn, N.Y., USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 7C is a side view of the main body 704 of vessel 701. A portion of vessel lid assembly 702 can be seen, as well as two impellers 706a and 706b. Also seen are a lactate/glucose sensor probe 734, a pH, O₂, CO₂ sensor 736 (such as a PRESENS™ integrated optical sensor (Precision Sensing GmbH, (Regensburg, Germany)), and a viable biomass sensor 738 (such as, e.g., the FUTURA PICO™ capacitance sensor (ABER, Alexandria, Va.)). In some embodiments, flat regions are fabricated onto the main body 704 of vessel 701 to reduce optical loss, simplify spot placement and simplify fluorescent measurement of pH, dO₂, and dCO₂.

FIG. 7D shows exemplary design guidelines for a one-impeller embodiment (left) and a two-impeller embodiment (right) of the main body 704 of vessel 701, including four exemplary impeller configurations. The embodiment of the INSCRIPTA™ bioreactor vessel 701 main body 704 as shown in this FIG. 7D has a total volume of 820 ml and supports culture volumes from 25 ml to 500 ml. As mentioned above, the impellers (and impeller shaft) may be injection molded or may be fabricated from stainless steel, other biocompatible metals, polymers or plastics and preferably comprised polished surfaces to facilitate sterilization. The impeller may be configured as a turbine-, pitched-blade-, hydrofoil- or marine-type impeller. In a two-impeller configuration, the impellers may be of the same type or different types. In the bioreactors described herein (the “INSCRIPTA™ bioreactors”), agitation is provided at 0-100 rpm, or 40-80 rpm, or approximately 70 rpm during cell growth (depending on the cell type being cultured); however, lower or higher revolutions per minute may be used depending on the volume of the main body 704 of vessel 701, the type of cells being cultured, whether the cells are adherent and being grown on microcarriers or the cells are non-adherent, and the size and configuration of the impellers. The impeller may turn in a clockwise direction, a counter-clockwise direction or the impeller may change direction (oscillate) or stop at desired intervals, particularly during cell detachment from the microcarriers. Also, intermittent agitation may be applied, e.g., agitating for 10 minutes every 30 minutes, or agitating for 1 minute every 5 minutes or any other desired pattern. Additionally, impeller rpm is often increased (e.g., up to 4000 rpm) when the cells are being detached from microcarriers. Although the present embodiment of INSCRIPTA™ bioreactor utilizes one or more impellers for cell growth, alternative embodiments of the INSCRIPTA™ bioreactor described herein may utilize bubbling or other physical mixing means.

Also seen in FIG. 7D is an equation that gives a range for exemplary bioreactor dimensions base on the height (H) and thickness (T) of the main body of vessel 704. For example, D=0.25−05*T means the impeller diameter could be one quarter or one half of the main body of vessel 704 thickness, T. C is the clearance of the impeller from the bottom of the main body of vessel 704, which can be 0.15 to 0.5 times the thickness. It should be apparent to one of ordinary skill in the art given the present disclosure that these numbers are just one embodiment and the ranges may be larger. The bioreactor vessel 701 main body 704 comprises an 8-10 mm clearance from the bottom of the main body 704 of vessel 701 to the lower impeller 706b and the lower impeller 706b and the upper impeller 706a are approximately 40 mm apart.

FIG. 7E is a side view of the vessel holder portion 714 of the bioreactor stand main body 712 of the bioreactor stand assembly 703. Inner surface 740 of vessel holder 714 is indicated and shown are camera or fiber optic ports 746 for monitoring, e.g., cell growth and viability; O₂ and CO₂ levels, and pH. The vessel holder portion 714 of the bioreactor stand main body 712 may also provide illumination using LED lights, such as a ring of LED lights (not shown). FIG. 7F is a side perspective view of the assembled bioreactor without sensors 742. Seen are vessel lid assembly 702, bioreactor stand assembly 703, bioreactor stand main body 712 into which the main body 704 of vessel 701 (not seen in FIG. 7E) is inserted. FIG. 7G is a lower side perspective view of bioreactor assembly 700 showing bioreactor stand assembly 703, bioreactor stand main body 712, vessel lid assembly 702 and two camera mounts 744. Surrounding bioreactor stand main body 712 is heater jacket 748.

FIGS. 7H-1 and 7H-2 together are an exemplary diagram of the bioreactor fluidics. Fluidics and pneumatics are designed to establish a cell culture environment conducive for mammalian cell growth, including iPSCs. Fluidic circuits are designed to deliver and/or remove cell medium, buffers, microcarriers and additional reagents needed for growth, maintenance, selection and passaging of the cells in the automated closed culture instrument. The pneumatic circuits are designed to deliver the appropriate gas mixture and humidity for the chosen cell type and may comprise line-in filters to prevent any contaminants from reaching the bioreactor.

FIG. 7I is a block diagram for an exemplary bioreactor control system. The control system is designed to control and automate the fluidics, pneumatics and sensor function in a closed system and without human intervention. In one embodiment, the control system is based on state-machines with a user editable state order and parameters using Json and jsonette config files. State-machines allow for dynamic control of several aspects of the bioreactor with a single computer.

In use, the bioreactor described herein is used for cell growth and expansion as well as for medium exchange and cell concentration. Medium/buffer exchange is in one embodiment accomplished using gravitational sedimentation and aspiration via a filter in the liquid-out port where the filter is of an appropriate size to retain microcarriers. In one embodiment used with the present bioreactor, a frit with pore size 100 μm was used and microcarriers with diameters or 120-225 μm were used in the cell culture. Sedimentation was accomplished in approximately 2-3 minutes for a 100 ml culture and 4-5 minutes for a 500 ml culture. The medium was aspirated at >100 ml/min rate. In addition to clearing the medium from the main body 504 of vessel 501, dead cells were removed as well. If sedimentation is used, the microcarriers do not typically accumulate on the filter; however, if accumulation is detected, the medium in the liquid-out port can be pushed back into main body 704 of vessel 701 in a pulse. In some embodiments—particularly those where sedimentation is not used—a cycle of aspiration, release (push back), aspiration and release (push back) may be performed.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Fully-Automated Singleplex RGN-Directed Editing Run

Singleplex automated genomic editing using MAD7 nuclease was successfully performed with an automated multi-module instrument of the disclosure. See U.S. Pat. No. 9,982,279; and U.S. Ser. No. 16/024,831 filed 30 Jun. 2018; Ser. No. 16/024,816 filed 30 Jun. 2018; Ser. No. 16/147,353 filed 28 Sep. 2018; Ser. No. 16/147,865 filed 30 Sep. 2018; and Ser. No. 16/147,871 filed 30 Jun. 2018.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example II: Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in an isothermal nucleic acid assembly module. The second editing vector comprised a kanamycin resistance gene, and the editing cassette comprised a galK_Y145* edit. If successful, the galK_Y145* edit confers on the cells the ability to uptake and metabolize galactose. The edit generated by the galK_Y154* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. Coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A method for editing a population of live cells with rationally-designed genome edits by replacing methylated nucleotide residues in genomes of the live cells with unmethylated residues or replacing unmethylated residues in genomes of the live cells with methylated residues and correlating the rationally-designed genome edits with resulting cellular nucleic acid profiles from individual cells in the population, wherein the method comprises the steps of:
designing and synthesizing a library of editing cassettes wherein each editing cassette comprises a repair template and a gRNA, wherein in some editing cassettes the repair template replaces genomic methylated nucleotide residues in genomes of the live cells with unmethylated residues and wherein in some editing cassettes the repair template replaces genomic unmethylated nucleotide residues in genomes of the live cells with methylated residues;
inserting the library of editing cassettes in a vector backbone resulting in a library of editing vectors;
transforming the population of cells with the library of editing vectors to produce transformed cells;
singulating the transformed cells into partitions;
allowing editing to take place in the singulated cells to produce edited cells;
lysing the edited cells;
conducting bisulfite conversion to convert unmethylated cytosine residues to uracil residues in the lysed cells;
adding barcoded random capture primers and barcoded cassette capture primers to each partition, wherein the barcodes used in the barcoded random capture primers and barcoded cassette capture primers in a same partition are a same barcode and wherein the barcodes used in the barcoded random capture primers and barcoded cassette capture primers in a different partition are different from barcodes used in other partitions;
creating DNA copies and/or cDNAs from cellular nucleic acids in the edited cells using the barcoded random capture primers;
creating DNA copies and/or cDNAs from the editing cassettes in the edited cells using the barcoded cassette capture primers;
pooling the DNA copies and/or cDNAs from the partitions;
sequencing the DNA copies and/or cDNAs;
correlating sequences from the DNA copies and/or cDNAs from cellular nucleic acids with sequences from the DNA copies and/or cDNAs from the editing cassettes; and
comparing the sequences from the DNA copies and/or cDNAs from cellular nucleic acids with a reference sequence to determine which cytosine residues in the cellular nucleic acids were converted to uracil residues for each cell.

2. The method of claim 1, wherein the sequencing step is performed by next generation sequencing.

3. The method of claim 1, wherein the live cells are grown in an automated cell processing instrument.

4. The method of claim 3, wherein the live cells are grown in a rotating growth module.

5. The method of claim 3, wherein the live cells are grown in a tangential flow filtration module.

6. The method of claim 3, wherein the live cells are grown and transformed in a bioreactor module.

7. The method of claim 6, wherein the live cells are grown and transformed on microcarriers.

8. The method of claim 6, wherein the live cells are grown in Accellta™ medium.

9. The method of claim 1, wherein the cells are singulated into droplets having barcoded random capture primers and barcoded cassette capture primers.

10. The method of claim 1, wherein the cells are singulated into wells having barcoded random capture primers and barcoded cassette capture primers.

11. The method of claim 8, wherein the wells are in a solid wall isolation incubation and normalization (SWIIN) module.

12. The method of claim 1, wherein the live cells are mammalian cells.

13. The method of claim 12, wherein the mammalian cells are iPSCs.

14. The method of claim 12, wherein the mammalian cells are primary cells.
